SEQUENCE LISTING (6 sequences)

SEQ ID NO: 1 - DNA, 25 bp, Artificial
  Numb FWD primer
gaggttcctacaggcacctgcccag

SEQ ID NO: 2 - DNA, 25 bp, Artificial
  Numb REV primer
caaaatcacccctcacagtactctg

SEQ ID NO: 3 - DNA, 26 bp, Artificial
  HLA-C primer 3
gcaaggattacatcgccctgaacgag

SEQ ID NO: 4 - DNA, 26 bp, Artificial
  HLA-C primer 4
catcatagcggtgaccacagctccaa

SEQ ID NO: 5 - DNA, 20 bp, Artificial
  t-PA1 FWD primer
agacagtacagccagcctca

SEQ ID NO: 6 - DNA, 21 bp, Artificial
  t-PA1 REV primer
gacttcaaatttctgctcctc

(19) United States
(12) Patent Application Publication
(10) Pub. No.: US 2011/0256592 A1
(43) Pub. Date: Oct. 20, 2011

(54) USE OF CYCLODEXTRINS TO IMPROVE THE SPECIFICITY, SENSITIVITY AND YIELD OF NUCLEIC ACID AMPLIFICATION REACTIONS

(75) Inventors: Marie-Claire Beckers, Beyne-Heusay (BE); Philippe Cronet, Neupre (BE); Adeline Vitale, Liege (BE); Eric Collette, Blegny (BE); Arzu Gullukaya, Jemeppe S/M (BE)

(73) Assignee: EUROGENTEC S.A, Seraing (BE)

(21) Appl. No.: 13/139,234

(22) PCT Filed: Dec. 14, 2009
(86) PCT No.: PCT/EP09/67110

§ 371 Date: Jun. 10, 2011

(30) Foreign Application Priority Data
Dec. 12, 2009 (EP) PCT/EP2008/067435

(51) Int. Cl. C12P 19/34; C12N 9/12

(52) U.S. Cl. 435/91.5; 435/194

(57) ABSTRACT

The invention is directed to methods for in vitro DNA synthesis catalysed by a DNA polymerase using cyclodextrins. The invention also relates to methods, compositions and kits comprising cyclodextrins for the amplification of a nucleic acid. The use of cyclodextrins improves the specificity, sensibility and/or yield of the amplification reaction. The invention is related more particularly to kits, compositions and methods for carrying out PCR reactions comprising a cyclodextrin.

This invention relates to kits, compositions and methods for nucleic acid amplification. More specifically it relates to improving the specificity, sensitivity and yield of PCR methods and of variants of PCR methods.

The polymerase chain reaction (PCR) is a technique widely used in molecular biology, genetics, diagnostics, clinical laboratories, forensic science, environmental science, hereditary studies, paternity testing and many other applications. The aim of PCR technology is to amplify a target nucleic acid from an undetectable amount of starting material.

PCR is a powerful and sensitive technique for DNA amplification. PCR amplifies specific DNA sequences exponentially by using multiple cycles of a three-step process. First, the double-stranded DNA template is denatured at a high temperature. Sequence-specific primers are then annealed to sites, on opposite strands, flanking the target sequence. A thermostable DNA polymerase, such as Taq DNA polymerase, extends the annealed primers, thereby doubling the amount of the original DNA sequence. This newly synthesized product then becomes an additional template for subsequent cycles of amplification. These three steps are repeated for 20 to 35 cycles, resulting in a 10⁵-10⁹ fold increase in DNA concentration.

In spite of the huge success of PCR methods and PCR-related methods for amplification of nucleic acids there is still a need for improving specificity, sensitivity and yield of the reaction as well as performance of polymerases.

Some templates or target nucleic acids are difficult to amplify and/or yield non-specific side products such as primer dimers or oligomers and other double stranded side products containing joined primers. The production of unwanted non-specific side products may completely prevent the amplification of the target nucleic acid if this target nucleic acid is present in a very low concentration. This may be an acute problem in diagnostic kits resulting in false negative results.

The specificity of the amplification reaction is provided by the specific annealing of primers to the nucleic acid target at an optimized temperature which does not allow unspecific pairing. However, for various reasons, the reaction mixture may be held at a temperature lower than the ideal annealing temperature before the amplification is performed. When the reaction mixture is kept at lower temperatures the primers may undesirably bind to non-target nucleic acids in an unspecific manner. These byproducts can be amplified along with the target nucleic acid or can completely prevent the accurate and quantitative amplification of the target nucleic acid. This results in background and lessens the yield of the specific PCR product.

Physical blocks such as a wax barrier or wax beads can be used to separate the reaction components in a heat depended manner. However, a major drawback is that the melted barrier material remains in the reaction mixture for the duration of the PCR reaction.

A commonly used method for improving amplification reactions is HotStart PCR using a HotStart DNA polymerase. This technique allows the inhibition or blocking of the polymerase activity during the PCR reaction preparation. By limiting polymerase activity prior to PCR cycling, HotStart PCR reduces non-specific amplification and increases the PCR product yield. HotStart PCR is commonly performed by using reversible chemical modification of the DNA polymerase or by inhibition of the DNA polymerase with a specific antibody (EP 0 592 035, EP 0 771 870, EP 0 962 526). In both cases the inhibition of the DNA polymerase is reversible and the DNA polymerase is activated through an initial heating step, the so called “HotStart” which is carried out before the PCR cycles. However, these techniques require the use of a specifically prepared thermostable DNA polymerase and of a heating step.

WO2006/119419 describes materials for sequestering reagents in hot-start PCR wherein the sequestering agent is a polylactone matrix. A hot-start is required to release the PCR reagents from the polylactone matrix.

Cyclodextrins are cyclic oligosaccharides which have been the object of intense scrutiny primarily due to their ability to form so called “inclusion” complexes with other molecules called “guests”. Cyclodextrins generally comprise a cavity which can include the hydrophobic portion of a guest molecule. While the outer surfaces of the cyclodextrins are hydrophilic, the inner cavities are highly hydrophobic, making them capable of inclusion complex formation with a large variety of smaller hydrophobic molecules. The cavities have different diameters dependent on the number of glucose units. By forming inclusion complexes with various molecules or parts of molecules, they are able to alter the physiochemical properties of the guest molecule. This can lead to enhanced solubility of the guest molecules, e.g. active drug molecules, and increase their bioavailability. Poorly soluble drugs, rapidly deteriorating flavors, volatile fragrances or toxic molecules can be encapsulated. Cyclodextrins are used in the pharmaceutical industry as a mean to control the release of active ingredients in drug formulations. Moreover, cyclodextrins can stabilise labile molecules and protect them from degradation by light, temperature, oxidation, reduction and hydrolysis or by reducing their volatility. Therefore, cyclodextrins have found a number of applications in a wide range of fields including pharmacolgy, food industry and cosmetology. Because inclusion compounds of cyclodextrins with hydrophobic molecules are able to penetrate body tissues, these can be used to release biologically active compounds under specific conditions.

Cyclodextrins or cyclodextrin derivatives have been shown to catalyse certain chemical reactions.

Cyclodexytrins have also found some applications in molecular biology. WO91/02040 describes inclusion complexes comprising a fluorophore and a cyclodextrin for the labelling of ligands. The inclusion complex of the fluorophores with the cyclodextrin amplifies the signal. Primers labeled with these inclusion complexes are used for the sequencing of nucleic acids. WO00/37674 also describes the use of cyclodextrins in sequencing reactions to amplify the fluorescence of an excimer or exciplex label.

U.S. Pat. No. 5,705,345 describes methods and kits for preparing nucleic acids using cyclodextrin. This document discloses the use of cyclodextrins for neutralising extractants in DNA modification or amplification reactions. Cyclodextrins are for example effective in neutralisation of SDS or phenol.

EP 0 762 898 describes the inclusion of antisense oligonucleotides with cyclodextrins for therapeutical uses. WO95/32739 describes oligonucleotides complexed with a cyclodextrin for cellular delivery systems.

In molecular biology, cyclodextrins have been used to amplify fluorescence of labels and for delivery of therapeutic antisense oligonucleotides.

However, the use of cyclodextrins in nucleic acid amplification reactions has neither been described nor suggested in the prior art.

The present invention now surprisingly shows that the use of cyclodextrins improves the specificity, sensitivity and/or yield of DNA amplification reactions such as PCR reactions and variants of PCR reactions.

Moreover, cyclodextrins also improve the specificity, sensitivity and/or yield of isothermal DNA amplification reactions.

In a first embodiment, this improvement is obtained by pre-treating one of the components of the amplification reaction, such as the thermostable DNA polymerase, the dNTPs, or the primers, with cyclodextrin. Strikingly, in another embodiment, the improvement of the amplification reaction is also observed if the cyclodextrin is simply added to the final reaction mixture in which the nucleic acid amplification is performed.

The kits, compositions and methods of the present invention provide reduction of unspecific amplification products including primer dimers while the yield of specific target nucleic acid is improved.

The kits, compositions and methods of the present invention do not require a heat activation step (HotStart) but HotStart PCR techniques are further improved by the addition of cyclodextrins according to the present invention.

The overall efficiency, sensitivity and specificity of the nucleic acid amplification reaction are improved. Because the cyclodextrin may be simply added to the reaction mixture; the kits, compositions and methods according to the invention allow the practitioner a great flexibility. If pre-treatment with cyclodextrin of one of the components is carried out, the pre-treatment consist in a simple incubation with the cyclodextrin.

### Sequence Listing

SEQ ID No. 1: primer FWD NUMB
SEQ ID No. 2: primer REV NUMB
SEQ ID No. 3: primer HLA-C
SEQ ID No. 4: primer HLA-C
SEQ ID No. 5: primer FWD t-PA1
SEQ ID No. 6: primer REV t-PA1

### SUMMARY OF THE INVENTION

The present invention is related to methods for improving the yield, sensitivity and/or specificity of the amplification reaction of a target nucleic acid in a sample wherein the amplification reaction is performed in a final reaction mixture comprising at least one cyclodextrin.

In a first embodiment, the invention relates to methods for improving the yield, sensitivity and/or specificity of the amplification reaction of a target nucleic acid in a sample comprising the following steps:
a) Contacting the sample containing the target nucleic acid or suspected of containing the target nucleic acid with an amplification reaction mixture containing at least one cyclodextrin;
b) Performing the amplification reaction on the reaction mixture obtained in step a).

In a second embodiment, the invention relates to methods for improving the yield, sensitivity and/or specificity of the amplification reaction of a target nucleic acid in a sample comprising the following steps:
a) Contacting with a cyclodextrin at least one component selected from a thermostable DNA polymerase, a reaction buffer, dNTPs and primers;
b) Contacting the sample containing the target nucleic acid or suspected of containing the target nucleic acid with an amplification reaction mixture containing at least one component from step a);
c) Performing the amplification reaction on the reaction mixture obtained in step b).

In a third embodiment, the invention relates to methods for improving the yield, sensitivity and/or specificity of the amplification reaction of a target nucleic acid in a sample comprising the following steps:
a) Contacting the sample with a cyclodextrin to obtain a mixture of sample and cyclodextrin;
b) Contacting the mixture of sample and cyclodextrin with an amplification reaction mixture;
c) Performing the amplification reaction on the reaction mixture obtained in step b).

In a fourth embodiment, the invention relates to methods for improving the yield, sensitivity and/or specificity of the amplification reaction of a target nucleic acid in a sample comprising the following steps:
a) Contacting the sample with a an amplification reaction mixture;
b) Adding at least a cyclodextrin;
c) Performing the amplification reaction on the reaction mixture obtained in step b).

Preferably, the concentration of the cyclodextrin in the reaction mixture is comprised between 0.1 to 50 mM.

Preferably, the reaction mixture comprises between 0.01 and 0.2 units/μl of a thermostable DNA polymerase.

Preferably, the reaction mixture comprises at least a sample, a cyclodextrin, a thermostable DNA polymerase, a reaction buffer, dNTPs and at least one primer.

Preferably, the cyclodextrin is selected from the group consisting of α-cyclodextrins, β-cyclodextrins, γ-cyclodextrin and derivatives thereof.

More preferably, the cyclodextrin is selected from the group consisting of monopropanediamino-beta-cyclodextrin, 6-O-alpha-D-Maltosyl-beta cyclodextrin, hydroxypropyl-beta-cyclodextrin and 2-hydroxypropyl-beta-cyclodextrin.

Another object of the present invention is a method for improving the specificity and/or yield of in vitro synthesis of a nucleic acid catalyzed by a DNA polymerase comprising contacting a single stranded nucleic acid with a DNA synthesis reaction mixture comprising a DNA polymerase, a primer, dNTPs and at least one cyclodextrin.

Preferably, the method for improving the specificity and/or yield of in vitro synthesis of a nucleic acid catalyzed by a DNA polymerase comprises annealing of the primer to the single stranded nucleic acid and incorporating complementary dNTPs at the 3′ end of the primer.

Preferably, the concentration of the cyclodextrin in the final reaction mixture, comprising the single stranded nucleic acid and the DNA synthesis reaction mixture is comprised between 0.5 to 50 mM.

The present invention is also related to kits for amplification of a target nucleic acid in a sample comprising in the same container at least a cyclodextrin and at least one component selected from the group consisting of a thermostable DNA polymerase, a reaction buffer for nucleic acid amplification, dNTPs and oligonucleotide primers.

In some embodiments, the kits comprise, in the same or separate containers, a reverse transcriptase.

### DESCRIPTION OF THE INVENTION

The present invention is related to methods for improving the specificity, sensitivity and/or yield of in vitro synthesis of a nucleic acid catalyzed by a DNA polymerase comprising contacting a single stranded nucleic acid with a DNA synthesis reaction mixture comprising a DNA polymerase, a primer, dNTPs and at least one cyclodextrin. Sequencing and, any other reaction involving annealing of a primer followed by primer extension with a DNA polymerase, are improved when the reaction is carried out in the presence of at least one cyclodextrin.

The denaturation, annealing and elongation/extension steps may be repeated a desired number of times to amplify a target nucleic acid in a sample. Therefore, in a preferred embodiment, the invention provides methods for amplification of a nucleic acid using a cyclodextrin.

The present invention also relates to kits and compositions comprising a cyclodextrin. Advantageously, the invention provides kits and compositions for amplification of a nucleic acid. These kits and compositions are typically intended for research or for in vitro diagnostic applications.

The term “amplification” refers to in vitro methods for increasing the number of copies of a target nucleotide sequence in a sample. An amplification reaction usually consists of many rounds of repetitive temperature cycles allowing successive denaturation, annealing and primer extension cycles. However, isothermal amplification methods are also within the scope of the present invention. The invention provides methods, compositions and kits for carrying out PCR or a variant of the PCR reaction such as isothermal amplification. These methods are described in the literature and well known to the person skilled in the art.

Typically, PCR reactions involve a repetitive series of 20-35 thermal cycles comprising a denaturation step, a primer annealing step and an extension/elongation step. The reaction is commonly carried out in reaction volumes of 5-100 μl in small reaction tubes in a thermal cycler. The denaturation step allows complete denaturation of the nucleic acid at a temperature around 94° C.-95° C. This step yields single stranded DNA. The primer annealing step is commonly carried out at a temperature which is around 5° C. lower than the melting temperature of the primer-target sequence DNA duplex. At this step, the oligonucleotide primers bind specifically to the single stranded target sequence. The extension step is carried out around 72° C. but this depends on the DNA polymerase used. Taq DNA polymerase for example has its optimum at 72° C. At this step the DNA polymerase synthesizes a new DNA strand complementary to the target strand by primer extension adding dNTPs in 5′ to 3′ direction.

The invention relates both to the amplification of a DNA or RNA target nucleotide sequence. For the amplification of a RNA target a reverse transcriptase is used to obtain a DNA template.

The methods, kits and compositions of the present invention are useful for classical PCR, routine solution DNA/RNA quantification, Reverse-Transcriptase PCR (one and two steps), Real-Time PCR (Single labeled probes, Double-dye probes, Molecular Beacon probes, Scorpions probes, plexor primers, FRET probes, Padlock probes; dsDNA binding fluorescent entity which emits fluorescence only when bound to double stranded DNA (like SYBrGreen dye)), Nucleic Acid Sequence Based Amplification (NASBA), High-Resolution DNA Melt curve analysis (HRM), Multiplex Ligation-dependent Probe Amplification (MLPA), Real-time monitoring of thermophilic helicase-dependent amplification (tHDA), Primer extension, Rapid Amplification of cDNA Ends (RACE), Nested PCR, immuno-polymerase chain reaction (immuno-PCR), methods implicating Rolling circle replication (RCA), Chromatin ImmunoPrecipitation on Chip (ChIP on chip), applications using proximity ligation for detection of proteins, biomolecular interactions and singles copies of pathogens and solid phase based nucleic acid assays.

The kits, compositions and methods of the present invention are suitable for assays comprising both amplification and sequencing of a target nucleic acid. The kits, compositions and methods of the present invention are of particular use for diagnostic purposes, for genotyping and for SNP studies.

The nucleic acid containing or suspected of containing a target nucleotide sequence may be labelled or attached to a solid support such as beads or a solid surface. The oligonucleotide primers may also be labelled or attached to various supports including beads.

In the present invention, the purpose of the cyclodextrins in the reaction is not to amplify the signal of the label or marker such as the fluorescence of a label for example. Typically, the cyclodextrins do not include any label or marker. In the methods and kits of the present invention cyclodextrins are used to increase the yield, sensitivity and/or specificity of amplification reactions or of DNA synthesis reactions.

The kits, compositions and methods of the present invention provide for the amplification of a target nucleic acid.

In a preferred embodiment, the kits and compositions of the present invention comprise a cyclodextrin and at least another reagent required for amplification of a nucleic acid. The invention also relates to the use of a cyclodextrin in a method for amplification of a nucleic acid to improve the yield, the specificity and/or the sensibility of the reaction.

Cyclodextrins are a family of cyclic oligosaccharides. Common cyclodextrins are composed of 5 or more α-D-glucopyranoside units linked by α 1->4 glucosidic bonds. Cyclodextrins are well-known to the skilled person and may be produced for example from starch by means of enzymatic conversion. Surprisingly, the addition of a cyclodextrin improves the specificity, sensitivity and yield of nucleic acid amplification reactions.

Any cyclodextrin, modified cyclodextrin or any mixture thereof may be used in the kits, compositions and methods of the present invention.

α-cyclodextrin (six membered sugar ring molecule), β-cyclodextrin (seven sugar ring molecule), γ-cyclodextrin (eight sugar ring molecule) or derivatives thereof are preferred.

The general formulas of α-cyclodextrin, β-cyclodextrin and γ-cyclodextrin are shown below. These native cyclodextrins can serve as scaffolds on which functional groups and other substituents can be assembled. Substituted cyclodextrins or cyclodextrin derivatives can be used in the methods, kits and compositions of the present invention. The cyclodextrins may be modified by any substitution or functionalization of the hydroxyl groups with hydrophobic moieties or with ‘effecter groups’ e.g. saccharides or peptides. The formula of a modified β-cyclodextrin where R is organic radical is shown below. The general formula:

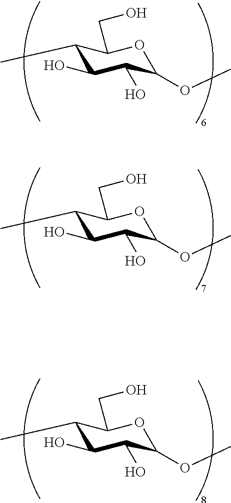

Modified β-cyclodextrins where R is an organic radical:

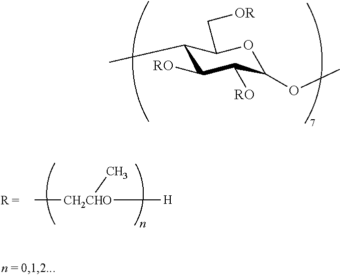

Any cyclodextrin or cyclodextrin derivative, providing the desired effect on the amplification reaction or on the nucleic acid synthesis reaction, may be used in the kits, compositions or methods of the present invention.

Among the α-cyclodextrins, preferred cyclodextrins include (2-hydroxypropyl)-alpha-cyclodextrin and 3A-amino-3A-deoxy-(2AS,3AS)-alpha-cyclodextrin hydrate.

Among the β-cyclodextrins, preferred cyclodextrins include monopropanediamino-beta-cyclodextrin (6′-(3-amino-propylamino)-6′-deoxy-cyclomaltoheptaose), 6-O-alpha-D-Maltosyl-beta-cyclodextrin, 2,6-Di-O-methyl-beta-cyclodextrin, hydroxyethyl-beta-cyclodextrin, (2-hydroxypropyl)-beta-cyclodextrin (beta cyclodextrin 2-hydroxypropylether), 3A-amino-3A-deoxy-(2AS,3AS)-beta-cyclodextrin hydrate and hydroxypropyl-beta-cyclodextrin.

In preferred embodiments, 2-hydroxypropyl-beta cyclodextrin is used, even more preferred 2-hydroxypropyl-beta cyclodextrin with a degree of substitution of between 0.5 and 0.7 hydroxypropyl groups per glucose unit is used. Most preferred, 2-hydroxypropyl-beta cyclodextrin with a degree of substitution of between 0.67 hydroxypropyl groups per glucose unit is used.

Among the γ-cyclodextrins, preferred cyclodextrins include (2-hydroxypropyl)-gamma-cyclodextrin and 3A-amino-3A-deoxy-(2AS,3AS)-gamma-cyclodextrin hydrate.

Reagents or components for nucleic acid amplification, including oligonucleotide primers, deoxyribonucleoside triphosphates (dNTPs), thermostable DNA polymerases and appropriate reaction buffers are described in the literature and known to one of ordinary skill in the art.

Any DNA polymerase may be used in the kits, compositions or methods of the present invention. Thermostable DNA polymerases are preferred. Preferred, thermostable DNA polymerases that may be used in the methods of the present invention include polymerases obtained from various Thermus bacterial species or from other microbial sources.

The preferred thermostable DNA polymerases are those obtainable from Thermus aquaticus, Thermus thermophilus, Thermus filiformis, Thermus flavus or Pyrococcus furiosus, woseii, and Thermococcus litoralis. These polymerases are preferably produced and purified from recombinant Escherichia coli which contain the gene encoding the DNA polymerase.

The kits, compositions and methods of the present invention may also use at least two thermostable DNA polymerases or a combination of a thermostable DNA polymerase with other enzymes such as Uracil-N-glycosylase, DNAse or an exonuclease such as a 3′-5′ proofreading exonuclease.

These enzymes or enzyme combinations can be especially useful for the amplification of long nucleic acid molecules.

Further, the kits, compositions and methods of the present invention may use a so-called HotStart DNA polymerase which has been reversibly inactivated by for example a chemical treatment or by a specific monoclonal antibody. These DNA polymerases require an initial heat activation step for 5-10 minutes at 90° C.-95° C.

Some reagents or components may be provided as a concentrated stock solution which is diluted upon preparation of the amplification reaction mixture for performing the reaction. Components may also be provided in a dry solid form, which is intended to be re-suspended in water or in an appropriate buffer, to prepare a stock solution.

The term “reaction buffer” refers to a buffering solution in which the enzymatic nucleic acid amplification is performed. The reaction buffer may be provided as a concentrated stock solution, typically in a 2×, 5× or 10× concentration. In the present invention, the reaction buffer may contain any known chemicals used in a buffer for nucleic acid amplification. The solution may for example contain Tris for buffering. Reaction buffers commonly also contain monovalent cations (KCl), divalent cations (MgC12, MgSO4) and non-ionic detergents (TritonX-100, Tween 20, NP40). The buffer may also contain reagents which enhance the PCR yield such as for example (NH4)2SO4, trehalose, DMSO, BSA, glycerol, MgC12, EDTA, betaine, etc. The reaction buffer may contain any cofactor required by the thermostable DNA polymerase. These reagents may be provided in the same container or in separate containers.

A standard 10×PCR buffer may for example comprise: 150-750 mM Tris-HCl pH 8-8.8, 50-200 nM (NH4)2SO4, 100-500 mM KCl, 0-20 mM MgCl2, 0.1% Tween-20 and 0.01% gelatin.

The term “storage buffer” refers to a buffering solution in which an enzyme such as a thermostable DNA polymerase is stored. This buffering solution may allow the storage of the enzyme at −20° C. or at 4° C. for several weeks or several months. Usually, the storage buffer contains glycerol for the storage of the enzyme at −20° C.

A standard 1× storage may for example comprise Tris 20 mM,EDTA 0.1 mM, KCl 100 mM, DTT 1-10 mM, Nonidet P40 0.50%, Tween-20 0.10-0.50%, Glycerol 0-50%, k-phospate 10 mM, Triton X-100 0.10%, PMSF 0.5 mM and Igepal CA-630 0.50%.

dNTPs include dATP, dCTP, dTTP and dGTP. The kits, compositions and methods of the present invention may also use modified or labelled dNTPs or nucleosides. dNTPs may be provided as a balanced stock solution of pre-mixed dATP, dCTP, dTTP and dGTP.

Pre-mixed dNTP is for example a solution in water of 5 mM of each 2′-deoxy-adenosine-5′-triphosphate, 2′-deoxy-cytidine-5′-triphosphate, 2′-deoxy-guanosine-5′-triphosphate, 2′-deoxy-thymidine-5′-triphosphate.

The kits, compositions and methods of the present invention may also use dUTP. The pre-mixed dNTP/dUTP solution contains for example contains 5 mM of each dATP, dCTP, dGTP and 10 mM 2′-deoxy-uridine-5′-triphosphate (dUTP).

The term “primer” refers to an oligonucleotide or to a derivative thereof having or containing a sequence complementary to a target nucleic acid. The primers hybridize to the denatured target nucleic acid through base pairing to initiate the extension reaction catalyzed by the DNA polymerase. The kits, compositions and methods of the present invention preferably use at least two oligonucleotide primers flanking the target nucleic acid and hybridizing to opposite strands of the nucleic acid. The kits, compositions and methods of the present invention may use labelled or modified oligonucleotides.

The term “amplification reaction mixture” refers to a solution comprising some or all the required components to carry out the reaction except for the sample. This amplification reaction mixture is usually termed the “MasterMix”. The Mastermix consists of some or all the required components to carry out the reaction except for the sample. The Mastermix typically comprises the reaction buffer, a balanced mix of dATP, dCTP, dTTP and dGTP, a thermostable DNA polymerase and primers.

The term “reaction mixture” or “final reaction mixture” refers to a solution comprising all the required components to carry out the reaction including the sample. The reaction mixture is usually prepared by mixing a determined volume of the amplification reaction mixture with a determined volume of sample. The final reaction mixture comprises both the sample containing or suspected of containing the target nucleic acid and the amplification reaction mixture. The final reaction mixture typically has a volume comprised between 5 and 100 μl.

PCR reactions or more generally amplification reactions may also be carried out in the presence of dsDNA binding fluorescent entity which emits fluorescence only when bound to double stranded DNA (like SYBrGreen dye). This is especially useful for Real-time PCR applications or quantitative PCR.

The term “sample” refers to any solid or liquid material containing or suspected of containing the target nucleic acid. The sample may be purified nucleic acids, a biological sample such as a tissue sample, a biological fluid sample or a cell sample. The sample may be for example blood, urine, serum or saliva. The sample may contain solid or liquid material of human, plant, animal, bacterial or viral origin.

Reaction components for nucleic acid amplification are commonly commercialised as kits comprising at least one or more of the reagents/components necessary to carry out the amplification of a target nucleic acid.

A first object of the present invention is a method for improving the yield, sensitivity and/or specificity of the amplification reaction of a target nucleic acid in a sample wherein the amplification reaction is performed in a reaction mixture comprising at least one cyclodextrin.

The invention relates to methods for improving the yield, sensitivity and/or specificity of the amplification of a target nucleic acid in a sample comprising the step of contacting the sample containing the target nucleic acid or suspected of containing the target nucleic acid with an amplification reaction mixture containing a cyclodextrin.

The cyclodextrin and the other reagents/components required for nucleic acid amplification may be added separately to the sample to set up the final reaction mixture in which the amplification is performed. Alternatively, the cyclodextrin may be pre-mixed with other components for nucleic acid amplification and than contacted with the sample to perform the amplification reaction. Alternatively, the sample may be contacted with cyclodextrin prior to the addition of other components for nucleic acid amplification.

In another embodiment, the cyclodextrin is used to pre-treat a component of the nucleic acid amplification. A thermostable DNA polymerase, oligonucleotide primers or dNTPs may be pre-treated with cyclodextrin. The pre-treated component is used to prepare an amplification reaction mixture containing cyclodextrin or to prepare directly a final reaction mixture containing cyclodextrin.

The invention is directed to methods for improving the yield, sensitivity and/or specificity of the amplification reaction of a target nucleic acid in a sample comprising the following steps:
a) Contacting the sample containing the target nucleic acid or suspected of containing the target nucleic acid with an amplification reaction mixture containing at least one cyclodextrin;
b) Performing the amplification reaction on the final reaction mixture obtained in step a).

The present invention is also directed to method for improving the yield, sensitivity and/or specificity of the amplification reaction of a target nucleic acid in a sample comprising the following steps:
a) Contacting with a cyclodextrin at least one component selected from a thermostable DNA polymerase, a reaction buffer, dNTPs and primers;
b) Contacting the sample containing the target nucleic acid or suspected of containing the target nucleic acid with an amplification reaction mixture containing at least one component from step a);
c) Performing the amplification reaction on the final reaction mixture obtained in step b).

The present invention is further directed to methods for improving the yield, sensitivity and/or specificity of the amplification reaction of a target nucleic acid in a sample comprising the following steps:
a) Contacting the sample with a cyclodextrin to obtain a mixture of sample and cyclodextrin;
b) Contacting the mixture of sample and cyclodextrin with an amplification reaction mixture;
c) Performing the amplification reaction on the final reaction mixture obtained in step b).
The present invention is further directed to a method for improving the yield, sensitivity and/or specificity of the amplification reaction of a target nucleic acid in a sample comprising the following steps:
a) Contacting the sample with a an amplification reaction mixture;
b) Adding at least a cyclodextrin;
c) Performing the amplification reaction on the final reaction mixture obtained in step b).

In the methods according to the invention, the concentration of the cyclodextrin in the reaction mixture, consisting of the sample and of the amplification reaction mixture, is preferably comprised between 0.1 and 50 mM, more preferably between 0.5 and 50 mM. Preferably between 0.1, 0.5, 1, 2, 4, 5 mM to 10, 15, 20, 25, 30, 40 and 50 mM. The adequate concentration of cyclodextrin may depend on the cyclodextrin used in the methods according to the invention. Methods as described in the present application can be used to assess the best concentration of cyclodextrin to obtain improvement of the sensitivity, specificity and/or yield of amplification methods.

In preferred embodiments, the cyclodextrin is selected from the group consisting of α-cyclodextrins, β-cyclodextrins, γ-cyclodextrin and derivatives thereof. Even more preferably, the cyclodextrin is selected from the group consisting of monopropanediamino-beta-cyclodextrin, 6-O-alpha-D-Maltosyl-beta cyclodextrin, hydroxyethyl-beta-cyclodextrin hydroxypropyl-beta-cyclodextrin and 2-hydroxypropyl-beta-cyclodextrin.

The concentration of thermostable DNA polymerase is another factor which may determine the sensitivity, specificity and yield of nucleic acid amplification methods. In a preferred embodiment, the final reaction mixture, comprising the sample and the amplification reaction mixture, comprises between 0.01, 0.02, 0.03, 0.04 units/μl to 0.05, 0.075, 0.1 and 0.2 units/μl of a thermostable DNA polymerase. A unit is defined as amount of enzyme required to incorporate 10 nmol of dNTPs into acid-insoluble material in 30 minutes at 72° C.

The methods according to the present invention may further comprise a heating step to denaturate the nucleic acids and/or to activate a “Hot Start” DNA polymerase. This additional step is required if the thermostable DNA polymerase has been reversibly inactivated by a chemical modification or by a monoclonal antibody.

Advantageously, the methods of the present invention do not require such a heating step/activation step at 95° C.

In the methods according to the invention, the amplification reaction mixture may comprise a thermostable DNA polymerase which has been pre-treated with a cyclodextrin.

In the methods of the present invention, the amplification reaction mixture may comprise dNTPs which have been pre-treated with a cyclodextrin.

In the methods of the present invention, the amplification reaction mixture may comprise at least one primer which has been pre-treated with a cyclodextrin.

The pre-treatment of a reagent/component of the amplification reaction mixture with cyclodextrin is carried out by incubating said component with a sufficient concentration of cyclodextrin. This incubation may be carried out at room temperature or preferably at 4° C. Incubation for one hour is usually sufficient to obtain the adequate pre-treatment of the reagent with the cyclodextrin.

Alternatively, the cyclodextrin may be used to prepare the amplification reaction mixture to which the sample is added or to prepare the final reaction mixture for performing the reaction.

In a preferred embodiment, the amplification reaction mixture comprises a cyclodextrin, a thermostable DNA polymerase, a reaction buffer, dNTPs and oligonucleotide primers.

Methods for amplification of a target nucleic acid are described in the literature and well-known to the person skilled in the art. PCR is the standard technique for nucleic acid amplification but variants of the PCR methods may also be used in the methods according to the invention.

The methods of the present invention may further comprise an initial denaturation step which is carried out at 94-95° C. for initial and complete denaturation of the nucleic acid. The heating step typically takes 1, 2, 3, or 5-10 minutes. This initial step may also serve as a heat activation step for a HotStart DNA polymerase.

The kits, compositions and methods of the present invention improve the specificity, sensitivity and/or yield of nucleic acid amplification reactions. Other known techniques may be used in combination with the present invention to further improve the reaction. Some reagents such as DMSO, BSA, glycerol, trehalose, betaine have been reported to improve the reaction. Further improvement may be provided by the use of a so called HotStart DNA polymerase.

The invention further relates to compositions containing a cyclodextrin and at least one reagent for the amplification of nucleic acids. The compositions of the present invention may be an amplification reaction mixture ready for performing the reaction upon addition of the sample. Alternatively, the compositions according to the invention may be in the form of a concentrated stock solution or a concentrated storage solution if it comprises an enzyme.

The invention provides for compositions for in vitro DNA synthesis catalysed by a DNA polymerase comprising a cyclodextrin, and at least one component selected from the group consisting of a DNA polymerase, a reaction buffer for in vitro DNA synthesis, a dNTP and a primer.

The invention also encompasses a composition for amplification of a target nucleic acid in a sample comprising a cyclodextrin, and at least one component selected from the group consisting of a thermostable DNA polymerase, a reaction buffer for nucleic acid amplification, a dNTP and an oligonucleotide primer.

In a preferred embodiment, the composition according to the invention comprises a cyclodextrin and a thermostable DNA polymerase.

Preferably, the composition according to the invention comprises cyclodextrin, a thermostable DNA polymerase and a storage buffer. The storage buffer is adapted for the storage of the thermostable DNA polymerase at 4° C. or −20° C.

As a general matter, to improve the yield, sensitivity and/or specificity of the nucleic acid amplification it is preferable to provide the cyclodextrin in a sufficient concentration. The invention also relates to a composition comprising a cyclodextrin and at least one dNTP. Preferably, the composition comprises a balanced mix of dATP, dTTP, dCTP and dGTP. The composition is for example a concentrated stock solution comprising a balanced pre-mix of dNTPs and a cyclodextrin. Alternatively, the composition may comprise dATP, dCTP, dGTP, dUTP and cyclodextrin.

Further, the invention relates to a composition comprising a cyclodextrin and a reaction buffer for nucleic acid amplification.

In another embodiment, the composition comprises cyclodextrin and at least one primer. Preferably, the composition comprises cyclodextrin and at least two primers hybridizing to opposite strands at the 5′ and 3′ ends of the target nucleic acid.

In a preferred embodiment, the composition comprises cyclodextrin, a thermostable DNA polymerase, dNTPs and a reaction buffer for nucleic acid amplification.

The composition may further comprise a sample containing a target nucleic acid or suspected of containing a target nucleic acid.

In the compositions and more specifically in the final reaction mixture, consisting of the sample and of the amplification reaction mixture, the concentration of the cyclodextrin is preferably comprised between 0.1 and 50 mM, more preferably between 0.5 and 50 mM. Preferably between 0.1, 0.5, 1, 2, 4, 5 mM to 10, 15, 20, 25, 30, 40 and 50 mM.

In the compositions and more specifically in the final reaction mixture, comprising the sample and the amplification reaction mixture, the amount of thermostable DNA polymerase is preferably comprised between 0.01, 0.02, 0.03, 0.04 units/μl to 0.05, 0.075, 0.1 and 0.2 units/μl.

In another embodiment, the compositions are part of a kit for amplifying a target nucleic acid sequence in a sample.

Another object of the present invention is a kit comprising, in the same or separate containers, a cyclodextrin and at least one component selected from the group consisting of a DNA polymerase, a reaction buffer for in vitro DNA synthesis, dNTPs and primers. Preferably, the kit is for amplification of nucleic acids.

In a preferred embodiment, the invention relates to kits comprising, in the same or separate containers, a cyclodextrin and at least one component selected from the group consisting of a thermostable DNA polymerase, a reaction buffer for nucleic acid amplification, dNTPs and oligonucleotide primers.

In another preferred embodiment, the invention is directed to a kit for amplification of a target nucleic acid in a sample comprising in the same container at least a cyclodextrin and at least one component selected from the group consisting of a thermostable DNA polymerase, a reaction buffer for nucleic acid amplification, dNTPs and oligonucleotide primers.

In the kits of the present invention, the different reagents are provided in separate containers or as pre-mixes comprising several components. The components may be provided as concentrated stock solutions which have to be mixed and diluted before the nucleic acid amplification. The components may also be provided in a dehydrated, lyophilised or any other dry solid form intended for re-suspension in water or in an appropriate buffer.

The kits may further comprise any cofactor for DNA polymerases.

The kits may also comprise dsDNA binding fluorescent entity which emits fluorescence only when bound to double stranded DNA (like SYBRGreen).

In a first embodiment, the kits according to the invention comprise in the same container a cyclodextrin and a thermostable DNA polymerase.

Preferably, the kits according to the invention comprise in the same container a cyclodextrin, a thermostable DNA polymerase and a storage buffer. The storage buffer is specifically intended and adapted for storage of thermostable DNA polymerase.

The thermostable DNA polymerase, kept in the storage buffer containing cyclodextrin, is usually diluted before the amplification of the nucleic acid is performed.

In a second embodiment, the kits according to the invention comprise in the same container a cyclodextrin and at least one dNTP. Preferably, the container comprises a cyclodextrin and a balanced concentrated pre-mix of dATP, dCTP, dGTP and dTTP. This dNTP pre-mix containing a cyclodextrin is diluted before amplification of the nucleic acid. Alternatively, the container comprises a cyclodextrin and a mixture of dATP, dCTP, dGTP and dUTP.

In another embodiment, the kits according to the invention comprise in the same container a cyclodextrin and a reaction buffer for nucleic acid amplification. Preferably, the container contains concentrated reaction buffer and cyclodextrin. This stock solution is diluted to prepare the amplification reaction mixture and the final reaction mixture in which the nucleic acid amplification is carried out.

In a further embodiment, the kits according to the invention comprise in the same container a cyclodextrin and at least one primer. Preferably, the container may comprise a cyclodextrin and at least two or more primers hybridizing to opposite strands at the 5′ and 3′ ends of the target nucleic acid sequences to be amplified.

In another embodiment, the kits according to the invention comprise in the same container a cyclodextrin, a dsDNA binding fluorescent entity which emits fluorescence only when bound to double stranded DNA.

In another embodiment, the kits comprise in the same container a labelled probe hybridizing to the target nucleic acid and a cyclodextrin.

The kits of the present invention provide for the amplification of both DNA and

RNA. Therefore, the kits of the present invention may also comprise a reverse transcriptase. The reverse transcriptase or RNA dependant DNA polymerase is used to synthesize cDNAs from a RNA template; thereafter the resulting cDNAs are amplified. Preferred, reverse transcriptases include Mu-MLV reverse transcriptase and AMV reverse transcriptase.

In some embodiments, the kits of the present invention provide for isothermal amplification. The kits may therefore comprise an helicase.

In the kits of the present invention, the cyclodextrin may be provided as a concentrated stock solution in a separate container or as a mixture with other components or reagents. In a preferred embodiment, the final concentration of the cyclodextrin in the final reaction mixture is preferably comprised between 0.1 and 50 mM, more preferably between 0.5 and 50 mM and even more preferably between 0.1, 0.5, 1, 2, 4, 5 mM to 10, 15, 20, 25, 30, 40 and 50 mM.

The kit according to the invention may contain the cyclodextrin in solution or in a dry solid form for re-suspension in water or in an appropriate buffer. The cyclodextrin may be provided as a pre-mix or concentrated stock solution with other components. The kit may also comprise instructions for preparation of the amplification reaction mixture having the appropriate concentration in cyclodextrin. The cyclodextrin may be directly added to the other reagents for preparation of the amplification reaction mixture. Alternatively, the cyclodextrin may be used to pre-treat one of the components of the reaction such as for example the thermostable DNA polymerase, the dNTPs or the oligonucleotide primers. In another embodiment, the cyclodextrin may be added to the sample containing or suspected of containing the target nucleic acid.

Preferably, an appropriate amount of thermostable DNA polymerase is used in the amplification reaction mixture which provides good specificity, yield and sensitivity. Preferably, the amount of thermostable DNA polymerase in the final reaction mixture is comprised between 0.01, 0.02, 0.03, 0.04 units/μl to 0.05, 0.075, 0.1 and 0.2 units/μl.

The kits of the present invention typically may not comprise all the components required for the amplification of nucleic acids. Some components such as the primers and the sample may be provided by the final user of the kit.

The kits of the present invention may also comprise an internal positive control (IPC). The internal positive control comprises primers and/or a specific probe and a control DNA template. This IPC may also contain cyclodextrin.

The invention is also related to the use of a kit comprising a cyclodextrin and, in the same or in separate containers, at least one reagent for the amplification of a nucleic acid.

### FIGURES

FIG. 1: Test of Taq polymerase+SA1 (cyclodextrin)

1:10 ng gDNA, Taq R-POL-P01 1×+SA1 4 mM; 2: 0 ng gDNA, Taq R-POL-P01 1×+SA1 4 mM; 3: 10 ng gDNA, Taq R-POL-P01 diluted 10x+SA1 4 mM; 4: 0 ng gDNA, Taq R-POL-P01 diluted 10×+SA1 4 mM; 5: 10 ng gDNA, Taq R-POL-P01 1×+dH2O; 6: 0 ng gDNA, Taq R-POL-P01 1×+dH2O; 7: 10 ng gDNA, Taq R-POL-P01 diluted 10x+dH2O; 8: 0 ng gDNA, Taq R-POL-P01 diluted 10×+dH2O; 9: 10 ng gDNA, GoldStar+SA1 4 mM; 10: 0 ng gDNA, GoldStar+SA1 4 mM; 11: 10 ng gDNA, GoldStar+dH2O; 12: 0 ng gDNA, GoldStar+dH2O; L: Smart ladder for small fragment.

FIG. 2: Test of Taq+SA1 (cyclodextrin) with HLA-C primers

L: Smart ladder; 1: 2 ng gDNA, TAQ without SA1; 2: NTC, TAQ without SA1; 3: 2 ng gDNA, TAQ+SA1 4 mM final; 4: NTC, TAQ+SA1 4 mM final;

FIG. 3: HLA-C amplified by PCR in Ignatov et al

FIG. 4: Test with Taq polymerase preparation with SA1 (cyclodextrin) and SAR (cyclodextrin)

L. Smart Ladder

1. Taq R-Pol-P01 with 10 ng gDNA; 2. Taq R-Pol-P01 with 1 ng gDNA; 3. NTC with Taq R-Pol-P01; 4. Taq R-Pol-P01 in presence of 9 mM SA1 with 10 ng gDNA; 5. Taq R-Pol-P01 in presence of 9 mM SA1 with 1 ng gDNA; 6. NTC with Taq R-Pol-P01 in presence of 9 mM SA1; 7. Taq R-Pol-P01 in presence of 9 mM SAR with 10 ng gDNA; 8. Taq R-Pol-P01 with 1 ng gDNA in presence of 9 mM SAR; 9. NTC with Taq R-Pol-P01 in presence of 9 mM SAR; 10. HotGoldStar with 10 ng gDNA; 11. HotGoldStar with 1 ng gDNA; 12. NTC with HotGoldStar 1. Taq R-Pol-P01 with 10 ng gDNA; 2. Taq R-Pol-P01 with 1 ng gDNA; 3. NTC with Taq R-Poi-P01; 4. Taq R-Pol-P01 in presence of 9 mM SA1 with 10 ng gDNA; 5. Taq R-Pol-P01 in presence of 9 mM SA1 with 1 ng gDNA; 6. NTC with Taq R-Pol-P01 in presence of 9 mM SA1; 7. Taq R-Pol-P01 in presence of 9 mM SAR with 10 ng gDNA; 8. Taq R-Pol-P01 in presence of 9 mM SAR with 1 ng gDNA; 9. NTC with Taq R-Pol-P01 in presence of 9 mM SAR; 10 HotGoldStar with 10 ng gDNA; 11. HotGoldStar with 1 ng gDNA; 12. NTC with HotGoldStar.

FIG. 5: Test of SAR in PCR with different Taq Polymerases

L. Smart Ladder SF

1-7. Taq Pol R-Pol-P01 with 10 ng gDNA without SaR

2-8. Taq Pol R-Pol-P01 with 1 ng gDNA without SaR

3-9. NTC with Taq Pol R-Pol-P01 without SaR

4-10. Taq Pol R-Pol-P01 with 10 ng gDNA in presence of 9 mM SaR

5-11. Taq Pol R-Pol-P01 with 1 ng gDNA in presence of 9 mM SaR

6-12. NTC with Taq Pol R-Pol-P01 in presence of 9 mM SaR

13-19. EGT GoldStar with 10 ng gDNA without SaR

14-20. EGT GoldStar with 1 ng gDNA without SaR

15-21. NTC with EGT GoldStar without SaR

16-22. EGT GoldStar with 10 ng gDNA in presence of 9 mM SaR

17-23. EGT GoldStar with 1 ng gDNA in presence of 9 mM SaR

18-24. NTC with EGT GoldStar in presence of 9 mM SaR

FIG. 6: Test of SAR in PCR with different Taq Polymerases

L. Smart Ladder SF

1-7. EGT HotGoldStar with 10 ng gDNA without SaR

2-8. EGT HotGoldStar with 1 ng gDNA without SaR

3-9. NTC with EGT HotGoldStar without SaR

4-10. EGT HotGoldStar with 10 ng gDNA in presence of 9 mM SaR

5-11. EGT HotGoldStar with 1 ng gDNA in presence of 9 mM SaR

6-12. NTC with EGT HotGoldStar in presence of 9 mM SaR

13-19. Roche FastStart with 10 ng gDNA without SaR

14-20. Roche FastStart with 1 ng gDNA without SaR

15-21. NTC with Roche FastStart without SaR

16-22. Roche FastStart with 10 ng gDNA in presence of 9 mM SaR

17-23. Roche FastStart with 1 ng gDNA in presence of 9 mM SaR

18-24. NTC with Roche FastStart in presence of 9 mM SaR

FIG. 7: Tests: dNTPs+SA1& Buffer+SA1

A (non-modified Taq R-POL-P07—GoldStar Cycling)

1. human gDNA 10 ng—Numb

2. human gDNA 1 ng—Numb

3. NTC—Numb

B. (non-modified Taq R-POL-P07—HotGoldStar Cycling)

4. human gDNA 10 ng—Numb

5. human gDNA 1 ng—Numb

6. NTC—Numb

C. (dNTP+SA1 10 mM in final reaction—GoldStar Cycling)

1. human gDNA 10 ng—Numb

2. human gDNA 1 ng—Numb

3. NTC—Numb

D. (dNTP+SA1 10 mM in final reaction—HotGoldStar Cycling)

4. human gDNA 10 ng—Numb

5. human gDNA 1 ng—Numb

6. NTC—Numb

E. (buffer reaction+SA1 10 mM final—GoldStar Cycling)

1. human gDNA 10 ng—Numb

2. human gDNA 1 ng—Numb

3. NTC—Numb

F. (buffer reaction+SA1 10 mM final—HotGoldStar Cycling)

4. human gDNA 10 ng—Numb

5. human gDNA 1 ng—Numb

6. NTC—Numb

G. (HotGoldStar—GoldStar Cycling)

1. human gDNA 10 ng—Numb

2. human gDNA 1 ng—Numb

3. NTC—Numb

H. (HotGoldStar—HotGoldStar Cycling)

4. human gDNA 10 ng—Numb

5. human gDNA 1 ng—Numb

6. NTC—Numb

FIG. 8: Tests: dNTPs+SA1 & Buffer+SA1

A (non-modified Taq R-POL-P07—GoldStar Cycling)

1. human gDNA 10 ng—tPA1

2. human gDNA 1 ng—tPA1

3. NTC—tPA1

B. (non-modified R-POL-P07—HotGoldStar Cycling)

4. human gDNA 10 ng—tPA1

5. human gDNA 1 ng—tPA1

6. NTC—tPA1

C. (dNTP+SA1 10 mM in final reaction—GoldStar Cycling)

1. human gDNA 10 ng—tPA1

2. human gDNA 1 ng—tPA1

3. NTC—tPA1

D. (dNTP+SA1 10 mM in final reaction—HotGoldStar Cycling)

4. human gDNA 10 ng—tPA1

5. human gDNA 1 ng—tPA1

6. NTC—tPA1

E. (buffer reaction+SA1 10 mM final—GoldStar Cycling)

1. human gDNA 10 ng—tPA1

2. human gDNA 1 ng—tPA1

3. NTC—tPA1

F. (buffer reaction+SA1 10 mM final—HotGoldStar Cycling)

4. human gDNA 10 ng—tPA1

5. human gDNA 1 ng—tPA1

6. NTC—tPA1

G. (HotGoldStar—GoldStar Cycling)

1. human gDNA 10 ng—tPA1

2. human gDNA 1 ng—tPA1

3. NTC—tPA1

H. (HotGoldStar—HotGoldStar Cycling)

4. human gDNA 10 ng—tPA1

5. human gDNA 1 ng—tPA1

6. NTC—tPA1

FIG. 9: PCR without initial heating step: Amplification with Taq PO8, Taq PO8+6 mM cyclodextrin and Hot GoldStar

### EXAMPLES

### Example 1

### Genes, Primers and Specific PCR Products

### 1. NUMB

### Primer Sequences: Numb

Primer FWD:
gag gtt cct aca ggc acc tgc cca g
Primer REV:
caa aat cac ccc tca cag tac tct g

The three bases of these primers can hybridize together and give at low temperature primer-dimers.

### NCBI Reference:

The target is identified as NT_{—}026437.11 in the NCBI database (sequence location: 54742877 to 54743182).

### Amplicon Length: 306 bp

### 2. HLA-C

HLA-C and the PCR fragment are described in “K. B. Ignatov, A. I. Miroshnikov, V. M. Kramarov, Russian Journal of Bioorganic Chemistry, 2003, vol. 29 (4), 368-371.”

### Primer Sequences: HLA-C

Primer 3:
gca agg att aca tcg ccc tga acg ag
Primer 4:
cat cat agc ggt gac cac agc tcc aa

Difficult template and very long PCR fragment to amplify

### NCBI Reference:

The target is identified as NT_{—}07592.14 in the NCBI database.

### Amplicon Length:

1230 bp

3. t-PA1
Primer Sequences: t-PA1

Primer FWD:
aga cag tac agc cag cct ca
Primer REV1:
gac ttc aaa ttt ctg ctc ctc

During PCR, in some conditions, these primers can generate the formation of primer-dimers.

### NCBI Reference:

The target is identified as NT_{—}007995.14 in the NCBI database.

### Amplicon Length: 374 bp

### Example 2

### Test of Taq Polymerase+SA1(Cyclodextrin)

gDNA: Human genomic DNA, Roche

Primers: Numb; amplicon=306 bp

GoldStar+kit. Ref: ME-0064-05 from Eurogentec (5 U/μl)

Taq polymerase: R-POL-P01 (Home made Taq)

2× Storage buffer:
Tris 40 mM; pH 8
EDTA 0.2 mM
KCl 0.2 M
DTT 2 mM
Nonidet P40 1% (v/v)
Tween-20 1%

### Cyclodextrin: SA1

C45H78N2O34+2HCl: Monopropanediamino-beta-cyclodextrin* or 6′-(3-amino-propylamino)-6′-deoxy-cyclomaltoheptaose (Chlorohydrated form).

### 1. DNA Preparation

gDNA Preparation

| | | Dilution to have a working |
|---|---|---|
| Sample | Stock conc. [ng/μl] | conc. of 10 ng/μl |
| Human gDNA | 200 | 1 μl gDNA stock + |
| | | 19 μl H₂O PCR grade |

For Tests, put 1 μl/25 μl of reaction

For NTC (negative control), put 1 μl of H2O PCR grade/25 μl of reaction

### 2. Polymerase Preparation

Taq R-POL-P01+SA1
a. Taq without dilution
   30 μl Taq+30 μl SA1 200 mM
   Shake and incubate 1 h at 4° C.
b. Taq diluted 10×
   3 μl Taq+27 μl storage buffer 2×+30 μl SA1 200 mM
   Shake and incubate 1 h at 4° C.
Taq R-POL-P01 alone
c. Taq without dilution
   5 μl Taq+5 μl H2O
d. Taq diluted 10×
   1 μl Taq+9 μl storage buffer 2×+10 μl H2O
GoldStar (5 U/μl)+SA1
1.25 μl GoldStar+3.75 μl Storage buffer 2×+5 μl SA1 200 mM
Shake and incubate 1 h at 4° C.
0.625 U/μl
GoldStar (5 U/μl) alone
1.25 μl GoldStar+3.75 μl Storage buffer 2×+5 μl H2O
0.625 U/μl
For tests and NTC, put 1 μl of enzyme/25 μl reaction

### 3. Mastermix Preparation

| | | |
|---|---|---|
| Components | Sample | Final conc. in 25 μl |

| 10x reaction buffer | 77.5 | μl | 1x | |
|---|---|---|---|---|
| dNTP 5 mM | 31 | μl | 200 | μM |
| Primer F 25 μM | 6.2 | μl | 200 | nM |
| Primer R 25 μM | 6.2 | μl | 200 | nM |
| MgCl2 25 mM | 62 | μl | 2 | mM |
| gDNA | (31 | μl) | 10 | ng/well |
| Polymerase | (31 | μl) | | |
| H2O | 530.1 | μl | | |
| Final volume | 775 | μl | | |

Put 23 μl of mastermix/25 μl reaction

### 4. Thermal Conditions

Thermal Cycler Conditions

10 min at 95° C.

35 cycles: 10 sec at 95° C.
5 sec at 60° C.
30 sec at 72° C.

5 min at 72° C.

4° C.∞

15 μl are loaded on gel (3 μl of blue+12 μl of template)

### 5. Results

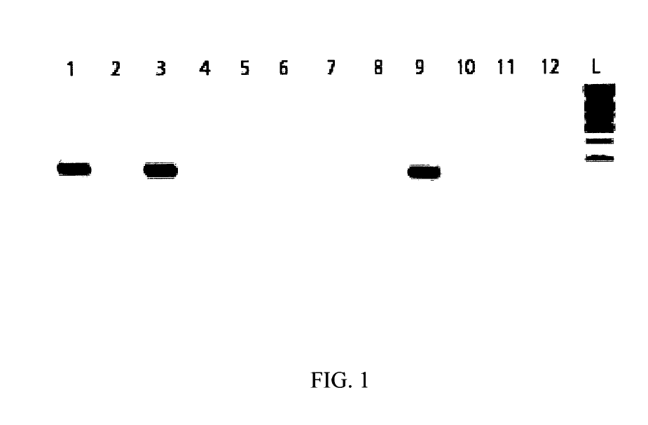

The results of the test of Taq polymerase+SA1 are shown in FIG. 1.

### 6. Conclusion

With Taq polymerase (1×) and SA1 there is amplification of the specific product from the Numb gene but also formation of primer-dimers.

With Taq polymerase (1×) without SA1, there's no amplification of the specific product from Numb but there is amplification of primer-dimers.

With Taq polymerase (diluted 10×) and SA1 there is amplification of the specific product from Numb but there is no formation of primer-dimers.

With Taq polymerase (diluted 10×) without SA1 there is only amplification of primer-dimers.

With GoldStar+SA1, there is amplification of the specific product from the Numb gene and no formation of primer-dimers.

With GoldStar without SA1 there is amplification of primer-dimers.

One explanation to these results: When the Taq polymerase is diluted 1×, there is an excess of TAQ by comparison with SA1. When Taq polymerase is diluted 10×, SA1 can interact with all Taq polymerase molecules and its effect is widespread to the whole reaction.

SA1 increases the specificity of the PCR reaction and decreases the formation of primer-dimers

### Example 3

### Test of Taq+SA1 (Cyclodextrin) with HLA-C Primers

### 1. Materials

### Enzyme: Home-made taq: Taq R-POL-P01

Primers HLA-C P3 and P4: more difficult template and long fragment to amplify

### Cyclodextrin: SA1

C45H78N2O34+2HCl Monopropanediamino-beta-cyclodextrin/6′-(3-amino-propylamino)-6′-deoxy-cyclomaltoheptaose (Chlorohydrated form)

### 2. Taq Polymerase Preparation

Prepare Taq Control directly before mastermix

| Taq R-POL-P01 | H2O PCR grade | Storage buffer 2x |
|---|---|---|
| 1 μl | 10 μl | 9 μl |

[symbol] Diluted 20×

1 μl/25 μl of reaction

Taq+SA1

Shake and incubate 1 h at 4° C.

| Taq R-POL-P01 | SA1 200 mM | Storage buffer 2x |
|---|---|---|
| 1 μl | 10 μl | 9 μl |

[symbol] Diluted 20×

1 μl/25 μl of reaction

### 3. DNA Preparation

gDNA preparation

| | | Dilution to have a working |
|---|---|---|
| Sample | Stock conc. [ng/μl] | conc. of 1 ng/μl |
| Human gDNA | 200 | 1 μl gDNA stock + |
| | | 199 μl H₂O PCR grade |

For Tests, put 2 μl/25 μl of reaction
For NTC, put 2 μl of H2O PCR grade/25 μl of reaction

### 4. Mastermix Preparation

Control—Taq+SA1 at 4 mM final

| | | |
|---|---|---|
| Components | Volume (μl) | Final concentration |

| | | | |
|---|---|---|---|
| gDNA | (16) | 2 | ng/reaction |
| 10x reaction buffer | 20 | 1 | x |
| dNTP 5 mM | 8 | 200 | μM |
| Taq R-POL-P01 | (8) | | |
| MgCl2 25 mM | 16 | 2 | mM |
| Primers HLA-C_P3 10 μM | 8 | 400 | nM |
| Primers HLA-C_P4 10 μM | 8 | 400 | nM |
| H₂O PCR grade | 116 | | |
| Final volume | 200 | | |

Put 22 μl/25 μl of reaction

### 5. Cycling

3 min at 95° C.

35 cycles: 30s at 94° C.
30 s at 58° C.
100 s at 72° C.

5 min at 72° C.

4° C. ∞

### 6. Results

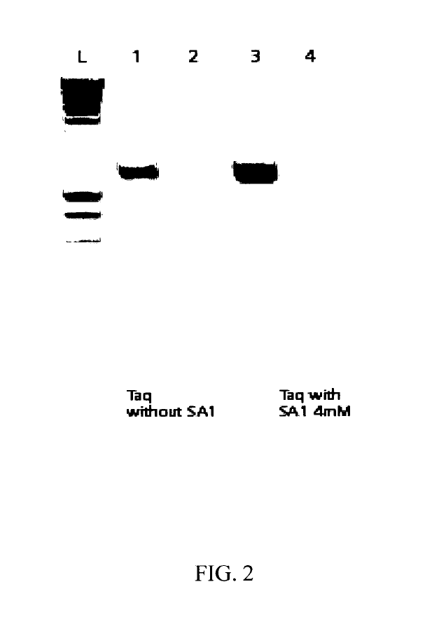

PCR results are shown in FIG. 2.

Quantification with Aida is shown below

| | | | Integral-Bkg | |
|---|---|---|---|---|
| Enzyme | gDNA | Integral-Bkg | no-specific | Integral-Bkg |
| conditions | quantity | HLA-C | products | Primer-dimers |
| Control | 2 ng | 1758 | 54.1 | 475.9 |
| | NTC | / | / | 511.2 |
| SA1 4 mM | 2 ng | 3280.1 | 137.4 | / |
| final | NTC | / | / | / |

### 7. Conclusion

The PCR reaction with the non-modified Taq polymerase shows specific fragment from HLA-C but also formation of primer-dimers and of non-specific product.

On the other hand, Taq-cyclodextrin preparation increases the amplification of specific PCR fragment and decreases strongly the non-specific product (no primer-dimers).

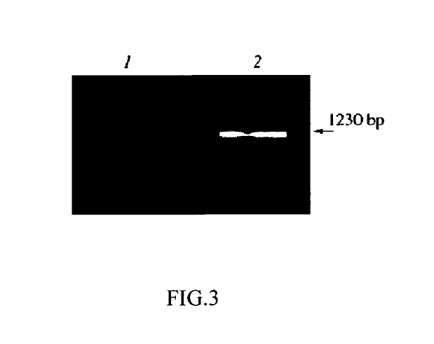

Amplification of HLA-C with these two primers has been described by Ignatov K. B. et al., Russian Journal of Bioorganic Chemistry, 29 (4), 2003, 368-371″ (see FIG. 3).

A hot-start method was used (see point 2).

A non-specific fragment is always seen on the gel.

### Example 4

### Test with Taq Polymerase Preparation with SA1 (Cyclodextrin) and SAR (Cyclodextrin)

PCR with primers Numb and HLA-C

### 1. Aim

Comparison tests with Taq prepared with SA1 and SAR at 9 mM final concentration in PCR.

### 2. Materials

Enzyme: Taq R-Pol-P01 home-made and HotGoldStar (Eurogentec Ref. ME-0073-05)
Cyclodextrin:
SA1: 6-(3-amino-propylamino)-6-deoxy-cyclomaltoheptaose/Monopropanediamino-beta-cyclodextrin, MW: 1264.02 g/mol
SAR: 6-O-alpha-D-Maltosyl-beta-cyclodextrin, MW: 1459.27 g/mol,
Primers: Numb, amplicon=306 bp
   HLA-C, amplicon=1230 bp
      3. Taq and SA1 preparation

### Control

| Taq R-POL-P01 | Storage buffer 2x |
|---|---|
| 2 μl | 18 μl |

SA1

SA1 250 mM: dilute 2× with H2O the SA1 stock 500 mM and stored at −20° C. This SA1 stock 500 mM was resuspended in 2× storage buffer.

→Mix 9 μl H2O PCR grade+9 μl SA1 500 mM and add 2 μl EGT Taq Pol.

| Taq R-POL-P01 | SA1 250 mM in Storage buffer 2x |
|---|---|
| 2 μl | 18 μl |
| | → SA1 diluted 1.11x |
| | Final concentration of SA1 is 225 mM |

SA1+Taq polymerase were shaken and incubated 15 min at 4° C. Add 1 μl/25 μl of PCR reaction.

SAR

SAR 250 mM: Weigh 0.0408 g SAR, add 56 μl 2× storage buffer and vortex to obtain a final concentration of 500 mM→the SAR isn't dissolved in the solution.

Add again 56 μl 2× storage buffer and vortex the SAR is totally dissolved, the final concentration is 250 mM.

| Taq R-POL-P01 | SAR 250 mM in Storage buffer 2x |
|---|---|
| 2 μl | 18 μl |
| | → SAR diluted 1.11x |
| | Final concentration of SA1 is 225 mM |

SAR+Taq polymerase were shaken and incubated 15 min at 4° C. Add 1 μl/25 μl of PCR reaction.

4. gDNA Preparation

| | | Dilution to have a working |
|---|---|---|
| Sample | Stock conc. [ng/μl] | conc. of 5 ng/μl |
| Human gDNA | 200 | 2 μl gDNA stock + |
| | | 78 μl H₂O PCR grade |

| | | Dilution to have a working |
|---|---|---|
| Sample | Stock conc. [ng/μl] | conc. of 0.5 ng/μl |
| Human gDNA | 5 | 6 μl gDNA stock + |
| | | 54 μl H₂O PCR grade |

For Tests, put 2 μl/25 μl of reaction
For NTC, put 2 μl of H2O PCR grade/25 μl of reaction

### 5. MasterMix Preparation

Prepare at room temperature (×8)

| Components | Volume (μl) | Final concentration | |
|---|---|---|---|
| DNA template | (8) | | |
| 10x reaction buffer | 10 | 1 | x |
| dNTPs 5 mM each | 4 | 200 | μM each |
| MgCl2 25 mM | 8 | 2 | mM |
| F Primer 10 μM | 2 | 200 | nM |
| R Primer 10 μM | 2 | 200 | nM |
| H2O PCR grade (Roche) | 62 | | |
| HotGoldStar | 65.5 | | |
| Taq R-POL-P01 (with | 4 | (9 | mM of SA1 or SAR) |
| 225 mM SA1 or SAr) | | | |
| HotGoldStar | 0.5 | 0.625 | U/reaction |
| Final volume | 100 | | |

Put 23 μl mastermix per well

Conditions with primers Numb and HLA-C:
MasterMix with EGT Taq Pol
MasterMix with EGT Taq Pol in presence of SA1 (mix 1:1)
MasterMix with EGT Taq Pol in presence of SAR (mix 1:1)
MasterMix with Hot GoldStar

### 6. Thermal Cycler Conditions (for HotGoldStar)

10 min at 95° C.
35 cycles: 10 sec at 95° C.
   10 sec at 60° C.
   30 sec at 72° C.
5 min at 72° C.
4° C. ∞

### 7. Results

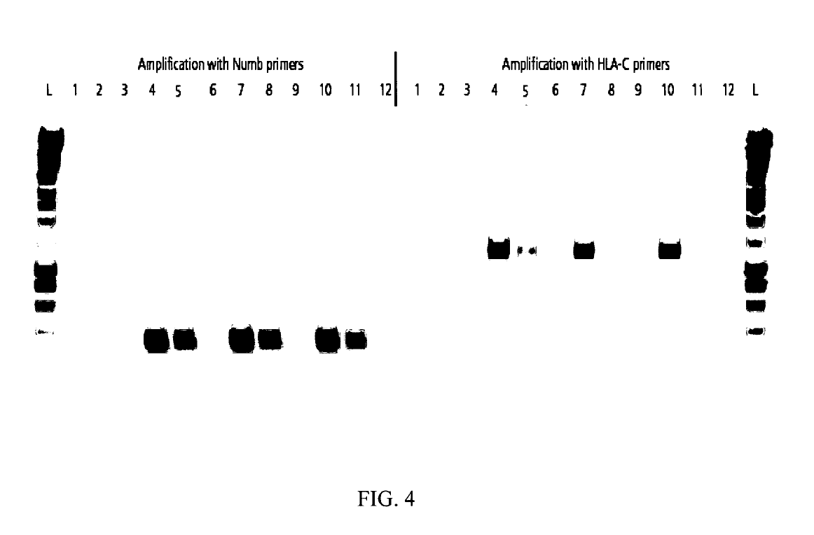

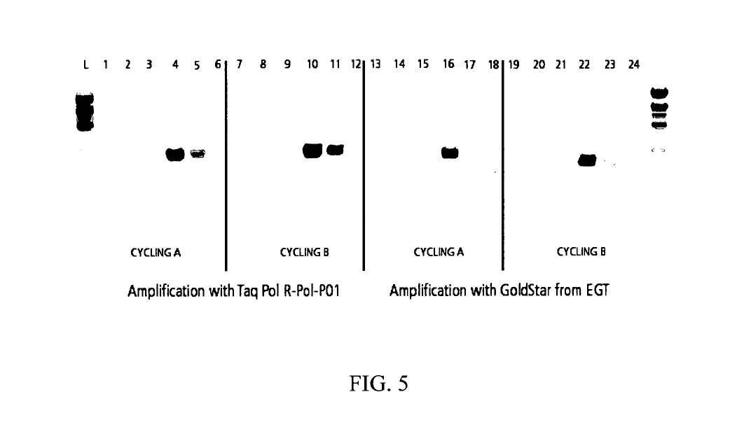

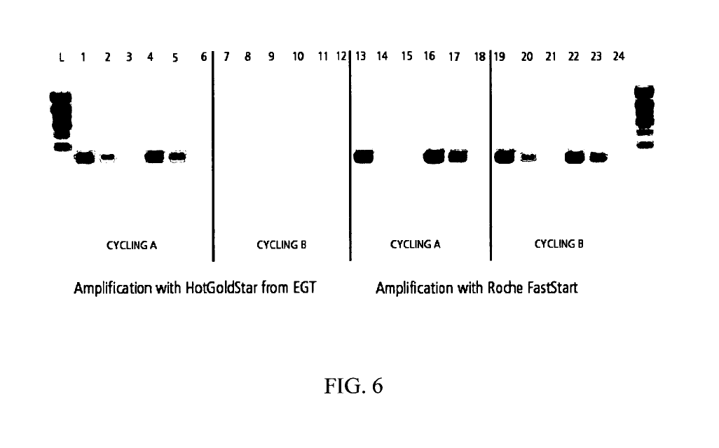

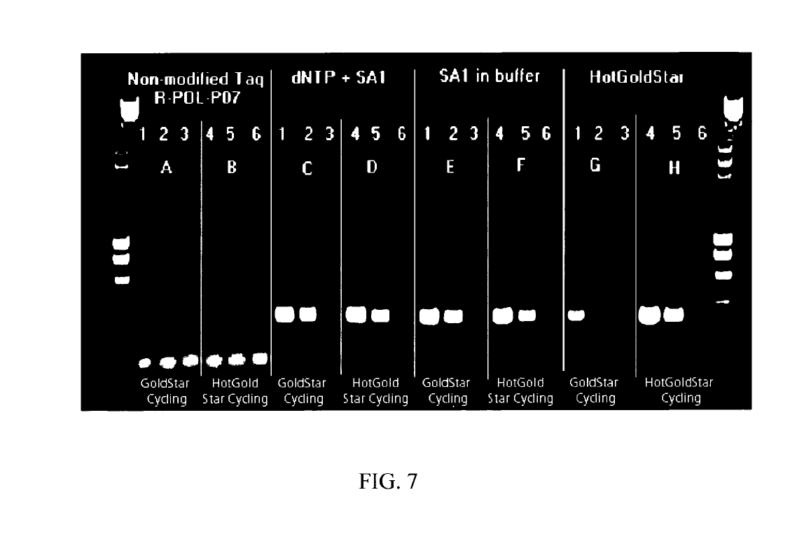

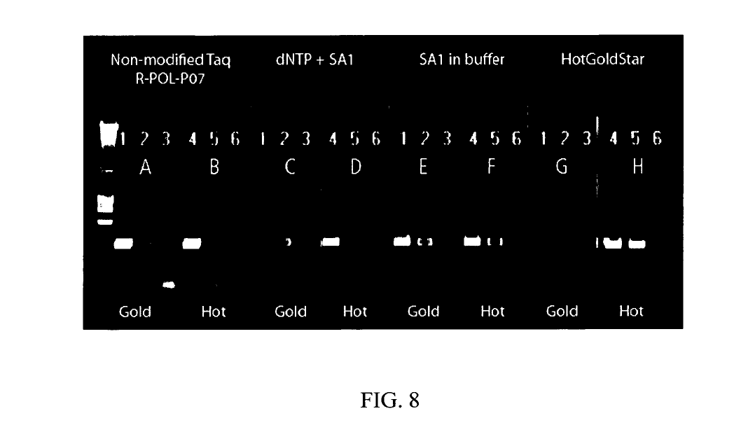

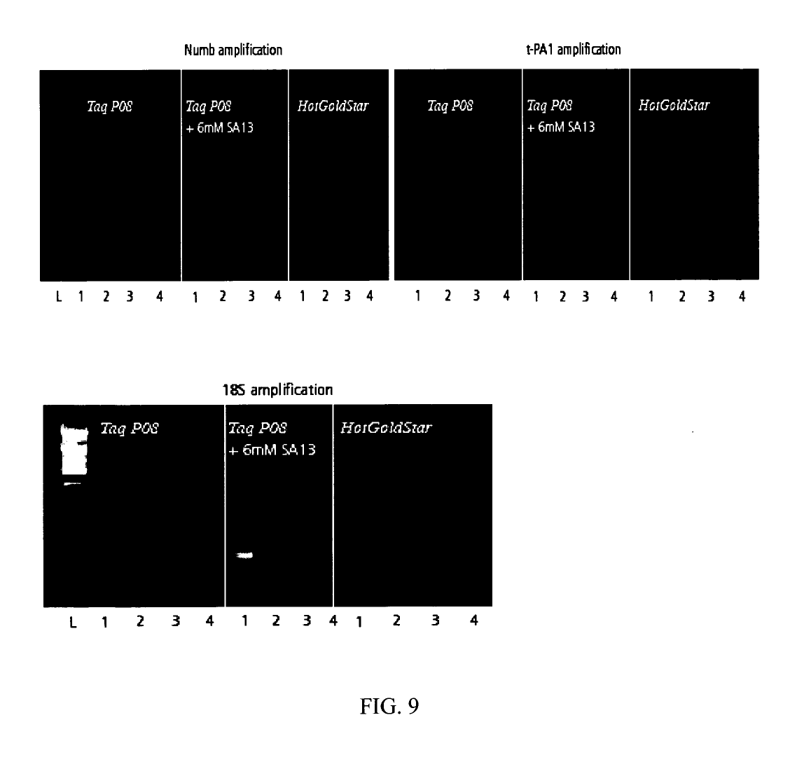

Results of the PCR reactions are shown in FIG. 4.

QC on 1% agarose gel

Quantification with Aida is shown below

| | | | | Integral- |
|---|---|---|---|---|
| | | | Integral- | Bkg |
| | Well | gDNA | Bkg | primer- |
| Enzyme conditions | number | quantity | Numb | dimers |
| Control with Numb | 1 | 10 ng | x | 896.56 |
| | 2 | 1 ng | x | 792.65 |
| | 3 | NTC | x | 371.54 |
| Taq + SA1 9 mM with | 4 | 10 ng | 22582.21 | x |
| Numb | 5 | 1 ng | 18110.4 | x |
| | 6 | NTC | x | x |
| Taq + SAR 9 mM with | 7 | 10 ng | 23544.29 | x |
| Numb | 8 | 1 ng | 18543.6 | 142.66 |
| | 9 | NTC | x | x |
| HotGoldStar with Numb | 10 | 10 ng | 22335.30 | 209.66 |
| | 11 | 1 ng | 11554.07 | 271.65 |
| | 12 | NTC | x | x |
| Control with HLA-C | 13 | 10 ng | 224.82 | 1110.84 |
| | 14 | 1 ng | x | 968.21 |
| | 15 | NTC | x | 899.17 |
| Taq + SA1 9 mM with | 16 | 10 ng | 19771.87 | x |
| HLA-C | 17 | 1 ng | 3186.39 | x |
| | 18 | NTC | x | x |
| Taq + SAR 9 mM with | 19 | 10 ng | 15448.48 | x |
| HLA-C | 20 | 1 ng | 1874.39 | x |
| | 21 | NTC | x | x |
| HotGoldStar with HLA-C | 22 | 10 ng | 16268.77 | x |
| | 23 | 1 ng | x | x |
| | 24 | NTC | x | x |

### 8. Conclusion

In the HotStar cycling conditions, these two different cyclodextrins used for the Taq polymerase preparations produced the same positive effect on the PCR results: more specific reaction and more yield.

### Example 5

### Test of SAR in PCR with Different Taq Polymerases

### 1. Aim

Test the following polymerases in presence or not of SAR (cyclodextrin) at 9 mM final concentration in PCR reaction: HotGoldStar, GoldStar, Taq Polymerase R-Pol-P01 and FastStart Taq DNA polymerase from Roche.

### 2. Materials

Enzyme: Taq Polymerase R-Pol-P01 home-made
HotGoldStar at 5 U/μl from Eurogentec (ref:ME-0073-05)
GoldStar at 5 U/μl from Eurogentec (ref:ME-0064-05)
FastStart Taq DNA Polymerase at 5 U/μl from Roche (ref: 12032902001)

10× reaction buffer to use with Taq POL R-Pol-P01 and GoldStar:
750 mM Tris/HCl pH 8.8 at 25° C., 200 mM (NH4)2SO4, 0.1% tween-20

10× reaction buffer to use with HotGoldStar:
150 mM Tris/HCl pH 8 at 25° C., 500 mM KCl, 0.1% tween-20

10×PCR reaction buffer from Roche to use with FastStart:
500 mM Tris/HCl, 100 mM KCl, 50 mM (NH4)2SO4, pH 8.3 at 25° C.

SAR: 6-O-alpha-D-Maltosyl-beta-cyclodextrin, MW: 1459.27 g/mol

Primers: Numb, amplicon=306 bp

### 3. Taq Polymerase and SAR Preparation

Control (Taq Polymerase without SAR)

| Taq Polymerase | Storage buffer 2x |
|---|---|
| 2 μl EGT Taq Pol. | 18 μl |
| 2 μl HotGoldStar | 18 μl |
| 2 μl GoldStar | 18 μl |
| 2 μl FastStart DNA Pol. (Roche) | 18 μl |

Taq polymerase with SAR

SAR stock at 250 mM

| Taq Polymerase | SAR in Storage buffer 2x |
|---|---|
| 2 μl EGT Taq Pol. | 18 μl SAR at 250 mM in storage buffer 2x |
| 2 μl HotGoldStar | 18 μl SAR at 200 mM in storage buffer 2x |
| 2 μl GoldStar | 18 μl SAR at 200 mM in storage buffer 2x |
| 2 μl FastStart DNA Pol. | 18 μl SAR at 200 mM in storage buffer 2x |
| (Roche) | |

SAR+Taq polymerase were shaken and incubated 15 min at 4° C.

4. gDNA Preparation

| | | Dilution to have a working |
|---|---|---|
| Sample | Stock conc. [ng/μl] | conc. of 5 ng/μl |
| Human gDNA | 200 | 2 μl gDNA stock + |
| | | 78 μl H₂O PCR grade |

| | | Dilution to have a working |
|---|---|---|
| Sample | Stock conc. [ng/μl] | conc. of 0.5 ng/μl |
| Human gDNA | 5 | 6 μl gDNA stock + |
| | | 54 μl H₂O PCR grade |

For Tests, put 2 μl/25 μl of reaction
For NTC, put 2 μl of H2O PCR grade/25 μl of reaction

### 5. MasterMix Preparation

Prepare at RT (×8)

| Components | Volume (μl) | Final concentration | |
|---|---|---|---|
| DNA template | (16) | | |
| 10x reaction buffer | 20 | 1 | x |
| dNTPs 5 mM each | 8 | 200 | μM each |
| MgCl2 25 mM | 16 | 2 | mM |
| F Primer 10 μM | 5 | 250 | nM |
| R Primer 10 μM | 5 | 250 | nM |
| H2O PCR grade (Roche) | 122 | | |
| For GoldStar, HotGolStar and | 120 | | |
| FastStart | | | |
| Taq R-POL-P01 | 8 | | |
| For GoldStar, HotGolStar and | 10 | 0.625 | U/reaction |
| FastStart | | | |
| Final volume | 200 | | |

Put 23 μl mastermix per well

Conditions with primers Numb and HLA-C:
MasterMix with EGT Taq Pol without SAR
MasterMix with EGT Taq Pol with SAR
MasterMix with GoldStar without SAR
MasterMix with GoldStar with SAR
MasterMix with HotGoldStar without SAR
MasterMix with HotGoldStar with SAR
MasterMix with fastStart Taq DNA polymerase without SAR
MasterMix with fastStart Taq DNA polymerase with SAR

### 6. Thermal Cycler Conditions

Cycling A—
10 min at 95° C.
35 cycles: 10 sec at 95° C.
   10 sec at 60° C.
   10 sec at 72° C.
5 min at 72° C.
4° C. ∞

Cycling B—
3 min at 95° C.
35 cycles: 10 sec at 95° C.
   10 sec at 60° C.
   30 sec at 72° C.
5 min at 72° C.
4° C. ∞

### 7. Results

QC on 1% agarose gel.

Amplification with Numb primers (Amplicon size=306 bp)

See results FIG. 5 and FIG. 6

Quantification with Aida is shown below

| | | | | Integral- |
|---|---|---|---|---|
| | | | Integral- | Bkg |
| | Well | gDNA | Bkg | primer- |
| Enzyme conditions | number | quantity | Numb | dimers |
| EGT Taq. Pol. without SAR | 1 | 10 ng | x | 16670.1 |
| Cycling A | 2 | 1 ng | x | 15479 |
| | 3 | NTC | x | 14236.7 |
| EGT Taq. Pol. with 9 mM | 4 | 10 ng | 27752.6 | 2656 |
| SAR | 5 | 1 ng | 15813.6 | 5693.4 |
| Cycling A | 6 | NTC | x | 11884.7 |
| EGT Taq. Pol. without SAR | 7 | 10 ng | x | 13141 |
| Cycling B | 8 | 1 ng | x | 16436.7 |
| | 9 | NTC | x | 15938.7 |
| EGT Taq. Pol. with 9 mM | 10 | 10 ng | 32170.8 | 3265.7 |
| SAR | 11 | 1 ng | 22000.8 | 7770.5 |
| Cycling B | 12 | NTC | x | 14944.2 |
| EGT GoldStar without SAR | 13 | 10 ng | x | 16646.7 |
| Cycling A | 14 | 1 ng | x | 11435.2 |
| | 15 | NTC | x | 10954.4 |
| EGT GoldStar with 9 mM | 16 | 10 ng | 22893.3 | 3259.6 |
| SAR | 17 | 1 ng | 10745 | 3995.3 |
| Cycling A | 18 | NTC | x | 2312.1 |
| EGT GoldStar without SAR | 19 | 10 ng | x | 16405.7 |
| Cycling B | 20 | 1 ng | x | 16270.2 |
| | 21 | NTC | x | 14165 |
| EGT GoldStar with 9 mM | 22 | 10 ng | 24111.5 | 3885.6 |
| SAR | 23 | 1 ng | 11366.9 | 3682.9 |
| Cycling B | 24 | NTC | x | 2457.9 |

Quantification with Aida is shown below

| | | | | Integral- |
|---|---|---|---|---|
| | | | Integral- | Bkg |
| | Well | gDNA | Bkg | primer- |
| Enzyme conditions | number | quantity | Numb | dimers |
| EGT HotGoldStar without | 1 | 10 ng | 16956.7 | 1084.7 |
| SAR | 2 | 1 ng | 7922.4 | 2230.6 |
| Cycling A | 3 | NTC | x | 2607.8 |
| EGT HotGoldStar with 9 mM | 4 | 10 ng | 17248 | x |
| SAR | 5 | 1 ng | 9476.1 | x |
| Cycling A | 6 | NTC | x | x |
| EGT HotGoldStar without | 7 | 10 ng | x | x |
| SAR | 8 | 1 ng | x | x |
| Cycling B | 9 | NTC | x | x |
| EGT HotGoldStar with 9 mM | 10 | 10 ng | x | x |
| SAR | 11 | 1 ng | x | x |
| Cycling B | 12 | NTC | x | x |
| Roche FastStart without SAR | 13 | 10 ng | 24806.6 | 487.4 |
| Cycling A | 14 | 1 ng | 948.9 | 3950.4 |
| | 15 | NTC | x | 4230.2 |
| Roche FastStart with 9 mM | 16 | 10 ng | 29581.2 | x |
| SAR | 17 | 1 ng | 22300 | 612.9 |
| Cycling A | 18 | NTC | x | x |
| Roche FastStart without SaR | 19 | 10 ng | 26251.7 | 881.5 |
| Cycling B | 20 | 1 ng | 7871.4 | 2648.9 |
| | 21 | NTC | x | 2857.2 |
| Roche FastStart with 9 mM | 22 | 10 ng | 22316.6 | 242.8 |
| SAR | 23 | 1 ng | 12430.2 | 338.6 |
| Cycling B | 24 | NTC | x | x |

### 8. Conclusion

The 4 tested Taq Polymerase preparations with the cyclodextrin SAR give the very good results in PCR reactions.

Taq Polymerase R-pol-P01 and GoldStar from Eurogentec:
In presence of SAR, the two Taq amplify the specific PCR fragment from Numb.
We can observe that the primer-dimers disappear when we use SAR.
Specific bands are more intense with cyling B (3 min at 95° C.) than cycling A (10 min at 95° C.).

HotGoldStar from Eurogentec:
The enzyme isn't activated with cycling B (3 min at 95° C.)
With cycling A (10 min at 95° C.), the bands are more intense in presence of SaR.
We obtain less primer-dimers in presence of SaR

Roche FastStart:
With cycling A and B, the bands are more intense in presence of SaR.
We obtain less primer-dimers in presence of SaR

### Example 6

### Tests: dNTPs+SA1& Buffer+SA1

Aim of experiment: Test the addition of SA1 to the dNTPs or into the buffer and the use of non-modified Taq (Taq polymerase without SA1) in the PCR reaction.

1. Master mix preparation

Enzymes:
Taq Home-made (R-POL-P07)
HotGoldStar from Eurogentec (Reference: ME-0073-05)
Cyclodextrin: SA1
C45H78N2O34+2HCl Monopropanediamino-beta-cyclodextrin/6′-(3-amino-propylamino)-6′-deoxy-cyclomaltoheptaose (Chlorohydrated form)
DNA to test: Human gDNA
Primers: NUMB and tPA1
Prepare at RT (×8) for Taq R-POL-P07, HotGoldStar Taq

| Components | Volume (μl) | Final concentration | |
|---|---|---|---|
| DNA template | (16) | | |
| 10x reaction buffer | 20 | 1 | x |
| GoldStar buffer for Taq R-POL-P07 | | | |
| HotGoldStar buffer for HotGoldStar | | | |
| Taq | | | |
| dNTPs 5 mM each | 8 | 200 | μM each |
| MgCl2 25 mM | 16 | 2 | mM |
| F Primer 10 μM | 5 | 250 | nM |
| R Primer 10 μM | 5 | 250 | nM |
| H2O PCR grade (Roche) | 129 (125) | | |
| Taq R-POL-P07 | (5) | | |
| HotGolStar | 1 | 0.625 | U/reaction |
| Final volume | 200 | | |

Put 23 μl mastermix per well

Prepare at RT (×8) for Taq EGT1+SA1 in dNTP

| Components | Volume (μl) | Final concentration | |
|---|---|---|---|
| DNA template | (16) | | |
| 10x reaction buffer | 20 | 1 | x |
| GoldStar buffer for Taq R-POL-P07 | | | |
| HotGoldStar buffer for HotGoldStar | | | |
| Taq | | | |
| dNTPs 5 mM each (or dNTPs + SA1) | 16 | 200 | μM each |
| MgCl2 25 mM | 16 | 2 | mM |
| F Primer 10 μM | 5 | 250 | nM |
| R Primer 10 μM | 5 | 250 | nM |
| H2O PCR grade (Roche) | 121 (117) | | |
| Taq R-POL-P07 | (5) | | |
| HotGolStar | 1 | 0.625 | U/reaction |
| Final volume | 200 | | |

Put 23 μl mastermix per well

Prepare at RT (×8) for SA1 directly in buffer

| Components | Volume (μl) | Final concentration | |
|---|---|---|---|
| DNA template | (16) | | |
| 10x reaction buffer | 20 | 1 | x |
| GoldStar buffer for Taq R-POL-P07 | | | |
| HotGoldStar buffer for HotGoldStar | | | |
| Taq | | | |
| dNTPs 5 mM each | 8 | 200 | μM each |
| MgCl2 25 mM | 16 | 2 | mM |
| F Primer 10 μM | 5 | 250 | nM |
| R Primer 10 μM | 5 | 250 | nM |
| SA1 (500 mM) | 4 | | |
| H2O PCR grade (Roche) | 125 (121) | | |
| Taq R-POL-P07 | (5) | | |
| HotGolStar | 1 | 0.625 | U/reaction |
| Final volume | 200 | | |

Put 23 μl mastermix per well

2. Taq without SA1

SA1: 500 mM, freshly prepared.

0.3602 gr of SA1 in 569 μl of water.

Control non modified Taq

| Taq R-POL-P07 | Storage buffer 2x | glycerol |
|---|---|---|
| 3 μl | 117 μl | 120 μl |

dNTPs nreparation with SA1

| dNTPs | SA1 0.25M in water | Conc. SA1 in dNTPs |
|---|---|---|
| 100 μl | 100 μl | 0.125M |

dNTPs were incubated with SA1 1 h at 4° C.

3. gDNA Preparation

| | | Dilution to have a working |
|---|---|---|
| Sample | Stock conc. [ng/μl] | conc. of 5 ng/μl |
| Human gDNA | 200 | 2 μl gDNA stock + |
| | | 78 μl H₂O PCR grade |

| | | Dilution to have a working |
|---|---|---|
| Sample | Stock conc. [ng/μl] | conc. of 0.5 ng/μl |
| Human gDNA | 5 | 6 μl gDNA stock + |
| | | 54 μl H₂O PCR grade |

### 4. Thermal Cycler Condition (GenAmp PCR System 9700)

GoldStar Cycling

3 min at 95° C. (or 10 min at 95° C. for HotGoldStar Cycling)

35 cycles: 10 sec at 95° C.
20 sec at 60° C.
30 sec at 72° C.
10 min 72° C.
4° C. ∞

ABI 9700

### 5. Results

Results: agarose gel electrophoresis 1% (see FIGS. 7 and 8)

Quantification with Aida is shown below

| | | | | |
|---|---|---|---|---|
| Non-modified | 1 | 10 ng | 34150 | 240541 |
| Taq R-POL-P07 | 2 | 1 ng | X | 270019 |
| GoldStar cycling | 3 | NTC | X | 289590 |
| NUMB | | | | |
| Non-modified | 4 | 10 ng | 33234 | 228202 |
| Taq R-POL-P07 | 5 | 1 ng | X | 254001 |
| HotGoldStar cycling | 6 | NTC | X | 260652 |
| NUMB | | | | |
| dNTPs + SA1 | 1 | 10 ng | 196126 | 72827 |
| GoldStar cycling | 2 | 1 ng | 173296 | 75009 |
| NUMB | 3 | NTC | X | 60153 |
| dNTPs + SA1 | 4 | 10 ng | 206163 | 61240 |
| HotGoldStar cycling | 5 | 1 ng | 160783 | 101186 |
| NUMB | 6 | NTC | X | 99791 |
| SA1 in reaction buffer | 1 | 10 ng | 187851 | 71809 |
| GoldStar cycling | 2 | 1 ng | 154527 | 87657 |
| NUMB | 3 | NTC | | 116478 |
| SA1 in reaction buffer | 4 | 10 ng | 200178 | 98156 |
| HotGoldStar cycling | 5 | 1 ng | 153819 | 149741 |
| NUMB | 6 | NTC | X | 129271 |
| HotGoldStar | 1 | 10 ng | 112858 | 56920 |
| GoldStar cycling | 2 | 1 ng | 15018 | 58059 |
| NUMB | 3 | NTC | X | 41904 |
| HotGoldStar | 4 | 10 ng | 210721 | 52290 |
| HotGoldStar cycling | 5 | 1 ng | 172987 | 58688 |
| NUMB | 6 | NTC | | 58987 |
| Non-modified Taq | 1 | 10 ng | 176181 | X |
| Taq R-POL-P07 | 2 | 1 ng | 94649 | 87920 |
| GoldStar cycling | 3 | NTC | X | 145490 |
| t-PA1 | | | | |
| Non-modified | 4 | 10 ng | 172067 | X |
| Taq R-POL-P07 | 5 | 1 ng | X | 129078 |
| HotGoldStar cycling | 6 | NTC | X | X |
| t-PA1 | | | | |
| dNTPs + SA1 | 1 | 10 ng | 18377 | X |
| GoldStar cycling | 2 | 1 ng | 97844 | X |
| t-PA1 | 3 | NTC | X | X |
| dNTPs + SA1 | 4 | 10 ng | 175976 | X |
| HotGoldStar cycling | 5 | 1 ng | 73041 | X |
| t-PA1 | 6 | NTC | X | X |
| SA1 in reaction buffer | 1 | 10 ng | 210109 | X |
| GoldStar cycling | 2 | 1 ng | 122901 | X |
| t-PA1 | 3 | NTC | X | X |
| SA1 in reaction buffer | 4 | 10 ng | 193311 | X |
| HotGoldStar cycling | 5 | 1 ng | 112922 | X |
| t-PA1 | 6 | NTC | X | X |
| HotGoldStar | 1 | 10 ng | 35260 | X |
| GoldStar cycling | 2 | 1 ng | X | X |
| t-PA1 | 3 | NTC | X | X |
| HotGoldStar | 4 | 10 ng | 198896 | X |
| HotGoldStar cycling | 5 | 1 ng | 172051 | X |
| t-PA1 | 6 | NTC | X | X |

### 6. Conclusions

In presence of SA1 in dNTPs (10 mM in the final reaction), we observe a good amplification of Numb with few primer-dimer (with the non-modified Taq R-POL-P07 without SA1 in dNTPs there are a poor amplification and a lot of primer-dimers).
In presence of SA1 in reaction buffer (10 mM in final reaction), we observe a good amplification of Numb but a little more primer-dimer compared to the results obtained with SA1-dNTP preparation.
With HotGoldStar (in the HotGoldStar Cycling), we observe a good amplification of Numb and of tPA1.
Concerning t-PA1 amplification, there are no primer-dimer when SA1 is added in dNTP or reaction buffer.

The addition of cyclodextrin to dNTP or even directly to the PCR buffer, improves the efficiency of the PCR reaction, the specific fragment is amplified and there is not the formation of primer-dimers (or much less that them obtained without cyclodextrin).

### Example 7

### PCR Reaction Without Initial Heating Step

Aim: Perform PCR reaction without initial heating step at 95° C. with TAQ polymerase, TAQ+SA13 and HotGoldStar DNA polymerase

### Materials:

Enzyme:
Home-made taq: Taq R-Pol-P08
HotGoldStar at 5 U/μl (Eurogentec, ref: ME-0073-01)

SA13: Hydroxypropyl-β-cyclodextrin

Primers: Numb, amplicon=306 bp
18S, amplicon=121 bp
t-PA1, amplicon=374 bp

### Taq P08 Preparation:

Stock of Taq P08 diluted 20× in storage buffer.

| | Taq R-POL-P08 | |
|---|---|---|
| Solution | diluted 20x | SA in Storage buffer 2x |
| Without SA | 2.5 μl | 25 μl Storage buffer 2x |
| SA13 6 mM final | 2.5 μl | (SA13 at 300 mM) 25 μl |

SA+taq polymerase were shake and incubated 1 h at 4° C.
Add 1 μl of enzyme per 25 μl of PCR reaction

### EDNA Preparation:

| | | | to have a working |
|---|---|---|---|
| Sample | Stock conc. [ng/μl] | Dilution | conc. of |
| Human gDNA | 200 | 5 μl gDNA 200 ng/μl + 195 μl H₂O | 5 ng/μl |
| | 5 | 15 μl gDNA 5 ng/μl + 135 μl H₂O | 0.5 ng/μl |
| | 0.5 | 15 μl gDNA 0.5 ng/μl + 135 μl H₂O | 0.05 ng/μl |
| | 0.05 | 15 μl gDNA 0.5 ng/μl + 135 μl H₂O | 0.005 ng/μl |

### Mastermix Preparation:

Prepared at RT

| Components | Volume (μl) | Final concentration | |
|---|---|---|---|
| DNA template | (10) | | |
| 10x reaction buffer | 12.5 | 1 | x |
| dNTPs 5 mM each | 5 | 200 | μM each |
| MgCl2 25 mM | 10 | 2 | mM |
| F Primer 10 μM | 3.25 | 260 | nM |
| R Primer 10 μM | 3.25 | 260 | nM |
| H2O PCR grade (Roche) | 76 | | |
| | 80.37 | | |
| Taq R-POL-P08 | 5 | | |
| Hot GoldStar | 0.63 | 0.625 | U/reaction |
| Final volume | 125 | | |

Put 23 μl/25 μl of reaction

### Cycling:

PCR begins directly with cycles (no activation step):
35 cycles: 10 sec at 95° C.
   10 sec at 60° C.
   30 sec at 72° C.
5 min at 72° C.
4° C. ∞

Without Initial activation step 3 min 95° C. for Taq P08 and

Without Initial activation step 10 min at 95° C. for HotGoldStar

| | | | | Integral- |
|---|---|---|---|---|
| | | | Integral- | Bkg/1000 |
| | gDNA | | Bkg | primer- |
| Enzyme conditions | quantity | | Numb/1000 | dimers |
| Numb | 10 | ng | 3 | 116 |
| R-POL-P08 | 1 | ng | x | 123 |
| | 0.1 | ng | x | 131 |
| | NTC | | x | 128 |
| Numb | 10 | ng | 176 | 32 |
| R-POL-P08 + SA13 6 mM | 1 | ng | 94 | 33 |
| | 0.1 | ng | 5 | 41 |
| | NTC | | x | 89 |
| Numb | 1 | ng | 2 | 10 |
| Hot GoldStar | 0.1 | ng | x | 8 |
| | 0.01 | ng | x | 4 |
| | NTC | | x | 7 |
| t-PA1 | 10 | ng | 117 | 15 |
| R-POL-P08 | 1 | ng | 22 | 36 |
| | 0.1 | ng | x | 29 |
| | NTC | | x | 63 |
| t-PA1 | 10 | ng | 153 | 6 |
| R-POL-P08 + SA13 6 mM | 1 | ng | 80 | 3 |
| | 0.1 | ng | 4 | 3 |
| | NTC | | x | 5 |
| t-PA1 | 1 | ng | x | x |
| Hot GoldStar | 0.1 | ng | x | x |
| | 0.01 | ng | x | x |
| | NTC | | x | x |
| 18S | 1 | ng | 43 | 3 |
| R-POL-P08 | 0.1 | ng | 10 | 11 |
| | 0.01 | ng | x | 30 |
| | NTC | | x | 36 |
| 18S | 1 | ng | 95 | x |
| R-POL-P08 + SA13 6 mM | 0.1 | ng | 26 | 0.8 |
| | 0.01 | ng | 1 | 3 |
| | NTC | | x | 2 |
| 18S | 1 | ng | 4 | x |
| Hot GoldStar | 0.1 | ng | x | x |
| | 0.01 | ng | x | x |

| | | |
|---|---|---|
| NTC | x | x |
| NTC | x | x |

### CONCLUSION

HotGoldStar Taq polymerase requires an activation step at 95° C. before PCR cycling. Without this activation step, there are not amplification of specific genes with HotGoldSar. We observe an amplification of the specific genes with the non-modified TAQ polymerase and with TAQ—cyclodextrin preparation even if the initial heating step is removed. The addition of cyclodextrin to TAQ polymerase, improves the efficiency and sensitivity of the PCR reaction, the specific fragment is amplified and there is much less primer-dimers that them obtained without cyclodextrin.

### REFERENCES

K. B. Ignatov, et al., Russian Journal of Bioorganic Chemistry, 2003, vol. 29 (4), 368-371

### Patent References

EP 0 592 035
EP 0 771 870
EP 0 962 526
WO91/02040
WO00/37674
EP 0 762 898
WO 95/32739
U.S. Pat. No. 5,705,345
WO2006/119419

1.-16. (canceled)

17. A method for improving the yield, sensitivity and/or specificity of the amplification reaction of a target nucleic acid in a sample characterized in that the amplification reaction is performed in a reaction mixture comprising at least one cyclodextrin.

18. A method for improving the yield, sensitivity and/or specificity of the amplification reaction of a target nucleic acid in a sample according to claim 17 comprising the following steps:
a) Contacting the sample containing the target nucleic acid or suspected of containing the target nucleic acid with an amplification reaction mixture containing at least one cyclodextrin;
b) Performing the amplification reaction on the reaction mixture obtained in step a).

19. A method for improving the yield, sensitivity and/or specificity of the amplification reaction of a target nucleic acid in a sample according to claim 17 comprising the following steps:
a) Contacting with a cyclodextrin at least one component selected from a thermostable DNA polymerase, a reaction buffer, dNTPs and primers;
b) Contacting the sample containing the target nucleic acid or suspected of containing the target nucleic acid with an amplification reaction mixture containing at least one component from step a);
c) Performing the amplification reaction on the reaction mixture obtained in step b).

20. A method for improving the yield, sensitivity and/or specificity of the amplification reaction of a target nucleic acid in a sample according to claim 17 comprising the following steps:
a) Contacting the sample with a cyclodextrin to obtain a mixture of sample and cyclodextrin;
b) Contacting the mixture of sample and cyclodextrin with an amplification reaction mixture;
c) Performing the amplification reaction on the reaction mixture obtained in step b).

21. A method for improving the yield, sensitivity and/or specificity of the amplification reaction of a target nucleic acid in a sample according to claim 17 comprising the following steps:
a) Contacting the sample with an amplification reaction mixture;
b) Adding at least a cyclodextrin;
c) Performing the amplification reaction on the reaction mixture obtained in step b).

22. A method for improving the yield, sensitivity and/or specificity of the amplification reaction of a target nucleic acid in a sample according to claim 17 wherein the concentration of the cyclodextrin in the reaction mixture is comprised between 0.1 to 50 mM.

23. A method for improving the yield, sensitivity and/or specificity of the amplification reaction of a target nucleic acid in a sample according to claim 17 wherein the reaction mixture comprises between 0.01 and 0.2 units/μl of a thermostable DNA polymerase.

24. A method for improving the yield, sensitivity and/or specificity of the amplification reaction of a target nucleic acid in a sample according to claim 17 wherein the reaction mixture comprises at least a sample, a cyclodextrin, a thermostable DNA polymerase, a reaction buffer, dNTPs and at least one primer.

25. A method for improving the yield, sensitivity and/or specificity of the amplification reaction of a target nucleic acid in a sample according to claim 17 wherein the cyclodextrin is selected from the group consisting of α-cyclodextrins, β-cyclodextrins, γ-cyclodextrin and derivatives thereof.

26. A method for improving the yield, sensitivity and/or specificity of the amplification reaction of a target nucleic acid in a sample according to claim 17 wherein the cyclodextrin is selected from the group consisting of monopropanediamino-beta-cyclodextrin, 6-O-alpha-D-Maltosyl-beta cyclodextrin, hydroxyethyl-beta-cyclodextrin, hydroxypropyl-beta-cyclodextrin and 2-hydroxypropyl-beta-cyclodextrin.

27. A method for improving the specificity and/or yield of in vitro synthesis of a nucleic acid catalyzed by a DNA polymerase comprising contacting a single stranded nucleic acid with a DNA synthesis reaction mixture comprising a DNA polymerase, a primer, dNTPs and at least one cyclodextrin.

28. A method for improving the specificity and/or yield of in vitro synthesis of a nucleic acid catalyzed by a DNA polymerase according to claim 27 comprising annealing of the primer to the single stranded nucleic acid and incorporating complementary dNTPs at the 3′ end of the primer.

29. A method for improving the specificity and/or yield of in vitro synthesis of a nucleic acid catalyzed by a DNA polymerase according to claim 27 wherein the concentration of the cyclodextrin in the final reaction mixture, comprising the single stranded nucleic acid and the DNA synthesis reaction mixture is comprised between 0.1 to 50 mM.

30. A kit for amplification of a target nucleic acid in a sample comprising in the same container at least a cyclodextrin and at least one component selected from the group consisting of a thermostable DNA polymerase, a reaction buffer for nucleic acid amplification, dNTPs and oligonucleotide primers.

31. A kit for amplification of a target nucleic acid in a sample according to claim 30 further comprising, in the same or separate containers, a reverse transcriptase.

32. A composition for amplification of a target nucleic acid in a sample comprising a cyclodextrin, a thermostable DNA polymerase and a storage buffer.
